(19) United States
(12) Patent Application Publication
(10) Pub. No.: US 2024/0242785 A1
(43) Pub. Date: Jul. 18, 2024

(54) SYSTEM AND METHOD FOR DEVELOPING AN ALTERNATIVE DRUG THERAPY USING CHARACTERISTICS OF AN EXISTING DRUG THERAPY TO PRODUCE A SIMILAR PATHWAY BEHAVIOR

(71) Applicant: Inderpreet Jalli, Houston, TX (US)

(72) Inventor: Inderpreet Jalli, Houston, TX (US)

(21) Appl. No.: 18/617,165

(22) Filed: Mar. 26, 2024

Related U.S. Application Data
(63) Continuation-in-part of application No. 18/017,076, filed on Jan. 19, 2023.
(60) Provisional application No. 63/053,706, filed on Jul. 19, 2020.

(51) Int. Cl. G16C 20/30; G16B 15/30; G16C 20/70

(52) U.S. Cl. CPC G16C 20/30; G16B 15/30; G16C 20/70

(57) ABSTRACT

Systems and methods for determining effects of candidate therapies are provided. A reference model is obtained, for a biological network having nodes representing transitions between biological intermediates. The reference model includes a first subset of intervention nodes having reference intervention functions and constants for transitions responsive to reference therapy, and a first subset of control nodes having control functions for transitions absent reference therapy. A reference progression including a reference signature for an intermediate is produced using the reference model. For each candidate therapy, a candidate model for the network is determined, including a second subset of intervention nodes having candidate intervention functions and constants for transitions responsive to candidate therapy, and a second subset of control nodes having control functions for transitions absent candidate therapy. Candidate progressions including candidate signatures for the intermediate are generated using candidate models. Candidate therapies having signatures that match the reference signature are determined.

### CROSS REFERENCE TO RELATED APPLICATIONS

The present application is a continuation-in-part of U.S. patent application Ser. No. 18/017,076, filed Jan. 19, 2023, which is a national stage application under 35 U.S.C. 371 of International Application No. PCT/US21/42263, filed Jul. 19, 2021, which claims priority to U.S. Provisional Patent Application No. 63/053,706, filed Jul. 19, 2020, each of which is incorporated by reference herein, in its entirety, for all purposes.

### TECHNICAL FIELD

The present disclosure relates generally to systems and methods for evaluating candidate drug therapies using characteristics of a reference drug therapy to produce similar behavior.

### BACKGROUND

Today, drug therapies are used to treat pathogens and diseases. Drug therapies work by attacking along particular pathways of the pathogen. Generally speaking, a pathway is a causal chain of interactions that lead to the alteration of the normal functioning of a pathogen set off by a drug therapy chemically interacting with targetable biological elements of the pathogen.

While many drug therapies exist, much further exploration is being performed to find new drug therapies, either to fight pathogens for which no drug therapies yet exist or to replace current inadequate drug therapies. Drug therapies can be inadequate for a number of reasons.

First, some drug therapies do not cure a disease, but merely reduce prevalence or symptoms. Examples of such drug therapies ones used to fight HIV or herpes viruses. In both cases, while drug therapies reduce viral loads within a person, none of the drug therapies completely eliminate the virus.

Second, some drug therapies have side effects ranging from mild to severe, and in some cases, life-threatening. The biological elements targeted by drug therapies results to an altered pathway behavior of the targeted elements, and consequently, its interaction with the other biological elements as a result of a chain reaction. These altered pathway behaviors, however, can produce significant negative effects to the biological network. Moreover, the therapeutic molecule can interact with either known or unknown non-target elements in the network which can also produce a negative overall pathway behavior in the target network as above described.

Third, drug therapies are often prone to being resisted by evolving pathogens over time. Specifically, in the case of therapeutic agents that target pathogenic organisms such as bacteria, viruses, parasites, or even cancer cells, therapeutic agents can lose efficacy due to evolution in target populations. Resistance occurs when a subset of a targeted set of organisms or cells survive exposure due to a particular trait of that subset, and then pass on such resistant trait to a next generation.

Fourth, some drug therapies can be expensive to make. Drug synthesis is a multistep process, and one step can have considerable effects on the cost to manufacture a drug. For example, in 2011, 4-Phenyl-1, cost $260 to make just 50 grams.

A common strategy for rational computer-aided development of new drugs is to first identify new interactions between biological elements or entirely new biological elements that may be critical to cell function. Highly promising biological elements are then structurally characterized at the molecular level along with their interaction with potential therapeutic agents. The aim is to target a specific biological element that has a high probability of significantly altering the function of the target cell in the desired manner.

However, such methods pose significant problems. Discovery of new biological elements or interactions within known elements is extremely time consuming and resource-intensive, or filled with false positives for interaction results. A discovery of an interaction or biological element that is critical to cell function in initial lab tests is also unable to answer a central question: does disruption of the target biological element lead to the desired effect on the organism overall via the chain reaction mechanism?

### SUMMARY

Given the above background, there is an unmet need for improved therapies that cause a target effect on a target biological network. Moreover, there is an unmet need for such improved therapies to cause a target effect (e.g., avoid an adverse effect) on an off-target biological pathway.

What is needed in the art are systems and methods for designing improved drug therapies that achieve target effects on targeted and/or off-target biological networks, in particular, by using characteristics of an existing or a reference drug therapy to produce similar pathway behavior, thereby overcoming the above-identified problems in the art.

Advantageously, the present disclosure addresses the need in the art by providing systems and methods for determining an effect of a candidate drug therapy. The present disclosure further provides systems and methods for developing a new drug therapy using characteristics of an existing drug therapy. The present disclosure further provides systems and methods for finding a set of parameters for a new drug therapy such that the new drug therapy produces an outcome similar to an existing drug therapy.

One aspect of the present disclosure provides a method for determining an effect of a candidate drug therapy. In some embodiments, the method is performed at a computer system comprising one or more processors and a memory. In some embodiments, the method includes obtaining a reference off-target treatment model for an off-target biological network, where the off-target biological network comprises a first plurality of nodes and a first plurality of biological intermediates, each respective node in the first plurality of nodes representing a respective biological transition between two or more biological intermediates in the first plurality of biological intermediates, the reference off-target treatment model comprises a first subset of intervention nodes and a first subset of control nodes selected from the first plurality of nodes, each respective intervention node in the first subset of intervention nodes comprises (i) a corresponding reference intervention function for the respective biological transition, responsive to a reference drug therapy, at the respective intervention node, and (ii) a corresponding reference intervention constant, in a set of reference intervention constants, for the respective reference intervention function, and each respective control node in the first subset of control nodes comprises a corresponding control function for the respective biological transition, responsive to an absence of the reference drug therapy, at the respective control node.

In some embodiments, the method further includes producing, using the reference off-target treatment model, a corresponding reference time-course progression for the off-target biological network comprising at least a first reference signature for a first biological intermediate in the first plurality of biological intermediates. In some embodiments, the method further includes selecting a plurality of candidate drug therapies.

In some embodiments, the method further includes performing, for each respective candidate drug therapy in the plurality of candidate drug therapies, a procedure comprising: determining a respective candidate off-target treatment model for the off-target biological network, where the respective candidate off-target treatment model comprises a second subset of intervention nodes and a second subset of control nodes selected from the first plurality of nodes, each respective intervention node in the second subset of intervention nodes comprises (i) a corresponding candidate intervention function for the respective biological transition, responsive to the respective candidate drug therapy, at the respective intervention node, and (ii) a corresponding candidate intervention constant, in a set of candidate intervention constants, for the respective candidate intervention function, and each respective control node in the second subset of control nodes comprises a corresponding control function for the respective biological transition, responsive to an absence of the respective candidate drug therapy, at the respective control node. In some embodiments, the procedure further includes generating, using the respective candidate off-target treatment model, a corresponding candidate time-course progression for the off-target biological network comprising at least a first candidate signature for the first biological intermediate in the first plurality of biological intermediates.

In some embodiments, the method further includes determining, in the plurality of candidate drug therapies, one or more candidate drug therapies having a corresponding first candidate signature that matches the first reference signature. In some embodiments, the method further includes synthesizing, for each respective candidate drug therapy having a corresponding first candidate signature that matches the first reference signature, the respective candidate drug therapy. In some embodiments, the method further includes, after the synthesizing, using the one or more candidate drug therapies as treatment for a target biological network.

Another aspect of the present disclosure includes a computer system including one or more processors; memory; and one or more programs, where the one or more programs are stored in the memory and configured to be executed by the one or more processors, the one or more programs including instructions for performing any of the methods disclosed above.

Another aspect of the present disclosure includes a non-transitory computer readable storage medium storing one or more programs, the one or more programs comprising instructions, which when executed by an electronic device with one or more processors and a memory cause the electronic device to perform any of the methods disclosed above.

### BRIEF DESCRIPTION OF THE DRAWINGS

FIG. 1 illustrates an example schematic of a drug therapy interacting with a biological network, in accordance with some embodiments of the present disclosure.

FIG. 2 illustrates an exemplary reaction model of biological network, in accordance with some embodiments of the present disclosure.

FIG. 3 illustrates an example time-course progression of a biological network, specifically an actual time-course progression, in accordance with some embodiments of the present disclosure.

FIG. 4 illustrates an example mathematical model of a biological network, in particular a pre-treatment mathematical model, in accordance with some embodiments of the present disclosure.

FIG. 5 illustrates an example set of pretreatment node-velocity functions for a pre-treatment mathematical model of a biological network, in accordance with some embodiments of the present disclosure.

FIG. 6 illustrates an example pre-treatment time-course progression, in accordance with some embodiments of the present disclosure.

FIG. 7 illustrates an example existing-treatment reaction model, in accordance with some embodiments of the present disclosure.

FIG. 8 illustrates an example existing-treatment mathematical model, in accordance with some embodiments of the present disclosure.

FIG. 9 illustrates an example first set of existing-treatment intervention functions, in accordance with some embodiments of the present disclosure.

FIG. 10 illustrates an example set of existing-treatment intervention constants, in accordance with some embodiments of the present disclosure.

FIG. 11 illustrates an example set of existing-treatment intervention concentrations, in accordance with some embodiments of the present disclosure.

FIG. 12 illustrates example untreated node velocities, in accordance with some embodiments of the present disclosure.

FIG. 13 illustrates an example existing-treatment time-course progression, in accordance with some embodiments of the present disclosure.

FIG. 14 illustrates an example new-treatment reaction model, in accordance with some embodiments of the present disclosure.

FIG. 15 illustrates an example new-treatment mathematical model, in accordance with some embodiments of the present disclosure.

FIG. 16 illustrates an example second set of new-treatment intervention functions, in accordance with some embodiments of the present disclosure.

FIG. 17 illustrates an example set of new-treatment intervention constants, in accordance with some embodiments of the present disclosure.

FIG. 18 illustrates an example set of new-treatment intervention concentrations, in accordance with some embodiments of the present disclosure.

FIG. 19 illustrates an example second set of untreated node velocity functions, in accordance with some embodiments of the present disclosure.

FIG. 20 illustrates an example new-treatment time-course progression, in accordance with some embodiments of the present disclosure.

FIGS. 21A, 21B, and 21C illustrates an exemplary system topology for determining an effect of a candidate drug therapy, in accordance with some embodiments of the present disclosure.

FIGS. 22A, 22B, 22C, 22D, and 22E collectively provide a flow chart of processes and features for determining an effect of a candidate drug therapy, in which optional steps are indicated by dashed lines, in accordance with some embodiments of the present disclosure.

FIG. 23 illustrates an example schematic for developing a candidate drug therapy using characteristics of an existing drug therapy, in accordance with an embodiment of the present disclosure. P1: evaluation of reference drug therapy; P2: evaluation of candidate drug therapy; P3: de novo modeling of perturbation in targeted biological network; TBN: targeted biological network; non-TBN: non-targeted biological network (e.g., off-target biological network).

Like reference numerals refer to corresponding parts throughout the several views of the drawings.

### DETAILED DESCRIPTION

Given the above background, there is an unmet need for determining an effect of a candidate drug therapy, in particular, to obtain improved therapies that cause a target effect on a target biological network and/or an off-target biological pathway. Advantageously, the presently disclosed subject matter addresses the above-identified need in the art by providing systems and methods for using characteristics of an existing or a reference drug therapy to produce similar pathway behavior.

The present disclosure advantageously provides systems and methods for determining an effect of a candidate drug therapy.

In some embodiments, the method includes obtaining a reference off-target treatment model for an off-target biological network, where the off-target biological network comprises a first plurality of nodes and a first plurality of biological intermediates, each respective node in the first plurality of nodes representing a respective biological transition between two or more biological intermediates in the first plurality of biological intermediates. In some embodiments, the reference off-target treatment model comprises a first subset of intervention nodes and a first subset of control nodes selected from the first plurality of nodes. Each respective intervention node in the first subset of intervention nodes comprises (i) a corresponding reference intervention function for the respective biological transition, responsive to a reference drug therapy, at the respective intervention node, and (ii) a corresponding reference intervention constant, in a set of reference intervention constants, for the respective reference intervention function. Each respective control node in the first subset of control nodes comprises a corresponding control function for the respective biological transition, responsive to an absence of the reference drug therapy, at the respective control node.

In some embodiments, there is produced, using the reference off-target treatment model, a corresponding reference time-course progression for the off-target biological network comprising at least a first reference signature for a first biological intermediate in the first plurality of biological intermediates.

A plurality of candidate drug therapies is selected, and, for each respective candidate drug therapy in the plurality of candidate drug therapies, a respective candidate off-target treatment model for the off-target biological network is determined. In some embodiments, the respective candidate off-target treatment model comprises a second subset of intervention nodes and a second subset of control nodes selected from the first plurality of nodes. Each respective intervention node in the second subset of intervention nodes comprises (i) a corresponding candidate intervention function for the respective biological transition, responsive to the respective candidate drug therapy, at the respective intervention node, and (ii) a corresponding candidate intervention constant, in a set of candidate intervention constants, for the respective candidate intervention function. Each respective control node in the second subset of control nodes comprises a corresponding control function for the respective biological transition, responsive to an absence of the respective candidate drug therapy, at the respective control node.

In some embodiments, there is generated, using the respective candidate off-target treatment model, a corresponding candidate time-course progression for the off-target biological network comprising at least a first candidate signature for the first biological intermediate in the first plurality of biological intermediates. In some embodiments, one or more candidate drug therapies in the plurality of candidate drug therapies having a corresponding first candidate signature that matches the first reference signature are determined.

Reference will now be made in detail to embodiments, examples of which are illustrated in the accompanying drawings. In the following detailed description, numerous specific details are set forth to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

### Definitions

Unless defined otherwise, all technical and scientific terms used herein have the meaning commonly understood by a person skilled in the art to which this invention belongs. As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

As used herein, the term “subject” refers to any animal (e.g., a mammal), including, but not limited to, humans, and non-human animals (including, but not limited to, non-human primates, dogs, cats, rodents, horses, cows, pigs, mice, rats, hamsters, rabbits, and the like (e.g., which is to be the recipient of a particular treatment, or from whom cells are harvested). In certain embodiments, the subject is a human.

As used herein, the term “treating” or “treatment” refers to clinical intervention in an attempt to alter the disease course of the individual, cell, or biological network being treated and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, an “effective amount” or “therapeutically effective amount” is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse, or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

As used herein, the term “diagnosis” or “diagnosed” refers to a determination as to whether a subject is likely affected by a given disease, disorder or dysfunction. The skilled artisan will appreciate that a diagnosis can be made on the basis of one or more diagnostic indicators including but not limited to, for example, biomarkers, images, and/or symptoms, the presence, absence, or amount of which is indicative of the presence or absence of the disease, disorder or dysfunction.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

The terminology used in the present disclosure is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

### Example Systems for Determining an Effect of a Candidate Drug Therapy

One aspect of the present disclosure provides systems for determining an effect of a candidate drug therapy.

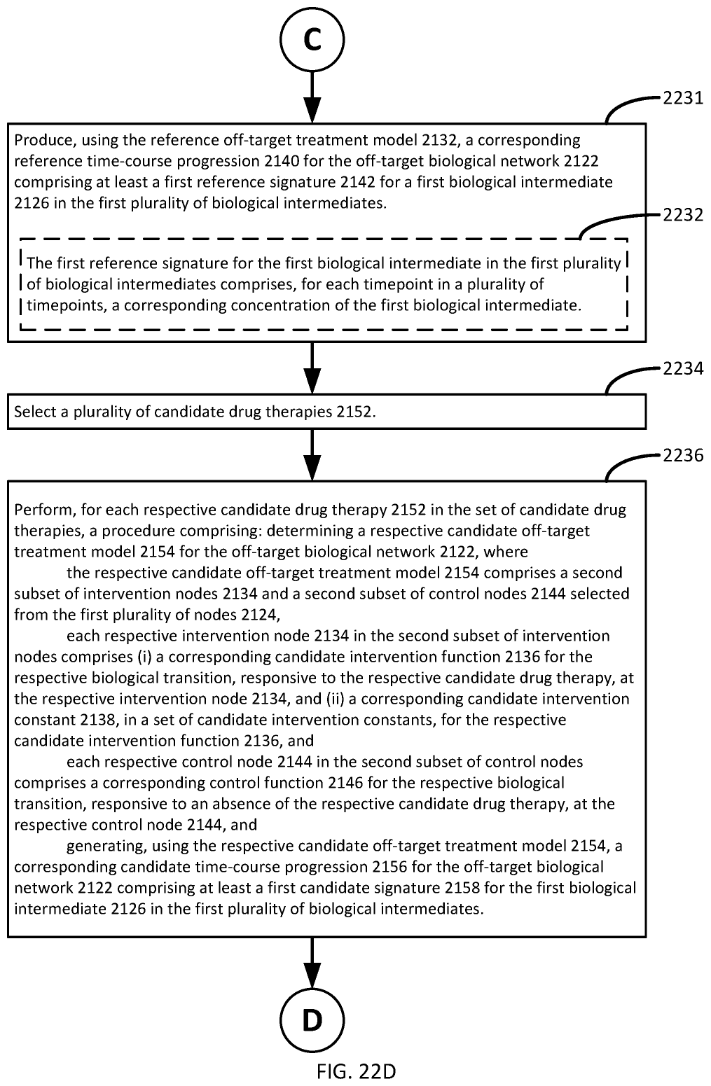

A detailed description of a system 100 for determining an effect of a candidate drug therapy, in accordance with the present disclosure, is described in conjunction with FIGS. 21A-C. As such, FIGS. 21A-C collectively illustrate the topology of the system in accordance with the present disclosure. In the topology, there is a system 100 for determining an effect of a candidate drug therapy.

Referring to FIGS. 21A-C, in some embodiments, the system 100 receives data directly or indirectly through radio-frequency signals. In some embodiments such signals are in accordance with an 802.11 (WiFi), Bluetooth, or ZigBee standard. In some embodiments the system 100 receives data across one or more communications networks 16.

Examples of networks 16 include, but are not limited to, the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of the present disclosure.

Of course, other topologies of the system 100 of FIGS. 21A-C are possible. For instance, rather than relying on a communications network 16, information may be sent directly to the system 100. Further, the system 100 may constitute a portable electronic device, a server computer, or in fact constitute several computers that are linked together in a network or be a virtual machine in a cloud computing context. As such, the exemplary topology shown in FIGS. 21A-C merely serves to describe the features of an embodiment of the present disclosure in a manner that will be readily understood to one of skill in the art.

Referring to FIGS. 21A-C, in typical embodiments, the system 100 comprises one or more computers. For purposes of illustration in FIGS. 21A-C, the system 100 is represented as a single computer that includes all of the functionality for performing any of the methods disclosed herein. However, the disclosure is not so limited. In some embodiments, the functionality is spread across any number of networked computers and/or resides on each of several networked computers and/or is hosted on one or more virtual machines at a remote location accessible across the communications network 16. One of skill in the art will appreciate that any of a wide array of different computer topologies are used for the application and all such topologies are within the scope of the present disclosure.

Turning to FIGS. 21A-C with the foregoing in mind, an exemplary system 100 for determining an effect of a candidate drug therapy comprises one or more processing units (CPUs) 74, a network or other communications interface 84, a memory 92 (e.g., random access memory), one or more magnetic disk storage and/or persistent devices 90 optionally accessed by one or more controllers 88, one or more communication busses 13 for interconnecting the aforementioned components, a user interface 78, the user interface 78 including a display 82 and input 80 (e.g., keyboard, keypad, touch screen), and a power supply 76 for powering the aforementioned components. In some embodiments, the input 80 is a touch-sensitive display, such as a touch-sensitive surface. In some embodiments, the user interface 78 includes one or more soft keyboard embodiments. The soft keyboard embodiments may include standard (QWERTY) and/or non-standard configurations of symbols on the displayed icons. In some embodiments, data in memory 92 is seamlessly shared with non-volatile memory 90 using known computing techniques such as caching. In some embodiments, memory 92 and/or memory 90 includes mass storage that is remotely located with respect to the central processing unit(s) 74. In other words, some data stored in memory 92 and/or memory 90 may in fact be hosted on computers that are external to the system 100 but that can be electronically accessed by the system 100 over an Internet, intranet, or other form of network or electronic cable (e.g., illustrated as element 16) using network interface 84.

In some embodiments, the memory 92 of the system 100 for determining an effect of a candidate drug therapy includes:
an optional operating system 2102 that includes procedures for handling various basic system services;
a biological network data store 2120, optionally comprising:
   an off-target biological network 2122 including a first plurality of nodes 2124 (e.g., 2124-1 . . . 2124-B) and a first plurality of biological intermediates 2126 (e.g., 2126-1 . . . 2126-C), each respective node 2124 in the first plurality of nodes representing a respective biological transition between two or more biological intermediates 2126 in the first plurality of biological intermediates, and
   an optional on-target biological network 2128;
a reference model construct 2130 comprising a reference off-target treatment model 2132, optionally including:
   a first subset of intervention nodes 2134 (e.g., 2134-ref-1 . . . 2134-ref-F), wherein each respective intervention node 2134 in the first subset of intervention nodes comprises (i) a corresponding reference intervention function 2136 (e.g., 2136-ref-1) for the respective biological transition, responsive to a reference drug therapy, at the respective intervention node 2134, and (ii) a corresponding reference intervention constant 2138 (e.g., 2138-ref-1), in a set of reference intervention constants, for the respective reference intervention function 2136, and
   a first subset of control nodes 2144 (e.g., 2144-ref-1 . . . 2144-ref-G), wherein each respective control node 2144 in the first subset of control nodes includes a corresponding control function 2146 (e.g., 2146-ref-1) for the respective biological transition, responsive to an absence of the reference drug therapy, at the respective control node 2144, and
   a corresponding reference time-course progression 2140 for the off-target biological network 2122, produced by the reference off-target treatment model 2132, including at least a first reference signature 2142 for a first biological intermediate 2126 in the first plurality of biological intermediates; and
a candidate therapy data store 2150, optionally comprising:
   a set (e.g., plurality) of candidate drug therapies 2152 (e.g., 2152-1 . . . 2152-J),
   for each candidate drug therapy 2152 in the set (e.g., plurality) of candidate drug therapies: a respective candidate off-target treatment model 2154 (e.g., 2154-1) for the off-target biological network 2122, including:
      a second subset of intervention nodes 2134 (e.g., 2134-cand-1-1 . . . 2134-cand-1-K), wherein each respective intervention node 2134 in the second subset of intervention nodes includes (i) a corresponding candidate intervention function 2136 (e.g., 2136-cand-1-1) for the respective biological transition, responsive to the respective candidate drug therapy, at the respective intervention node 2134, and (ii) a corresponding candidate intervention constant 2138 (e.g., 2138-cand-1-1), in a set of candidate intervention constants, for the respective candidate intervention function 2136,
      a second subset of control nodes 2144 (e.g., 2144-cand-1-1 . . . 2144-cand-1-L), each respective control node 2144 in the second subset of control nodes including a corresponding control function 2146 (e.g., 2146-cand-1-1) for the respective biological transition, responsive to an absence of the respective candidate drug therapy, at the respective control node 2144, and
      a corresponding candidate time-course progression 2156 (e.g., 2156-1) for the off-target biological network 2122, generated using the respective candidate off-target treatment model 2154, comprising at least a first candidate signature 2158 (e.g., 2158-1) for the first biological intermediate 2126 in the first plurality of biological intermediates.

In some implementations, one or more of the above identified data elements or modules of the system 100 are stored in one or more of the previously described memory devices, and correspond to a set of instructions for performing a function described above. The above-identified data, modules, or programs (e.g., sets of instructions) need not be implemented as separate software programs, procedures or modules, and thus various subsets of these modules may be combined or otherwise re-arranged in various implementations. In some implementations, the memory 92 and/or 90 optionally stores a subset of the modules and data structures identified above. Furthermore, in some embodiments the memory 92 and/or 90 stores additional modules and data structures not described above.

In some embodiments, the system 100 comprises a smart phone (e.g., an iPhone), laptop, tablet computer, desktop computer, or other form of electronic device (e.g., a gaming console). In some embodiments, the system 100 is not mobile. In some embodiments, the system 100 is mobile.

It should be appreciated that the system 100 illustrated in FIGS. 21A-C is only one example of a device that may be used, and that the system 100 optionally has more or fewer components than shown, optionally combines two or more components, or optionally has a different configuration or arrangement of the components. The various components shown in FIGS. 21A-C are implemented in hardware, software, firmware, or a combination thereof, including one or more signal processing and/or application specific integrated circuits.

In some embodiments, the system 100 has any or all of the circuitry, hardware components, and software components found in the system 100 depicted in FIGS. 21A-C. In the interest of brevity and clarity, only a few of the possible components of the system 100 are shown in order to better emphasize the additional software modules that are installed on the system 100.

While the system 100 disclosed in FIGS. 21A-C can work standalone, in some embodiments it can also be linked with electronic medical records to exchange information in any way.

### Example Embodiments for Determining an Effect of a Candidate Drug Therapy

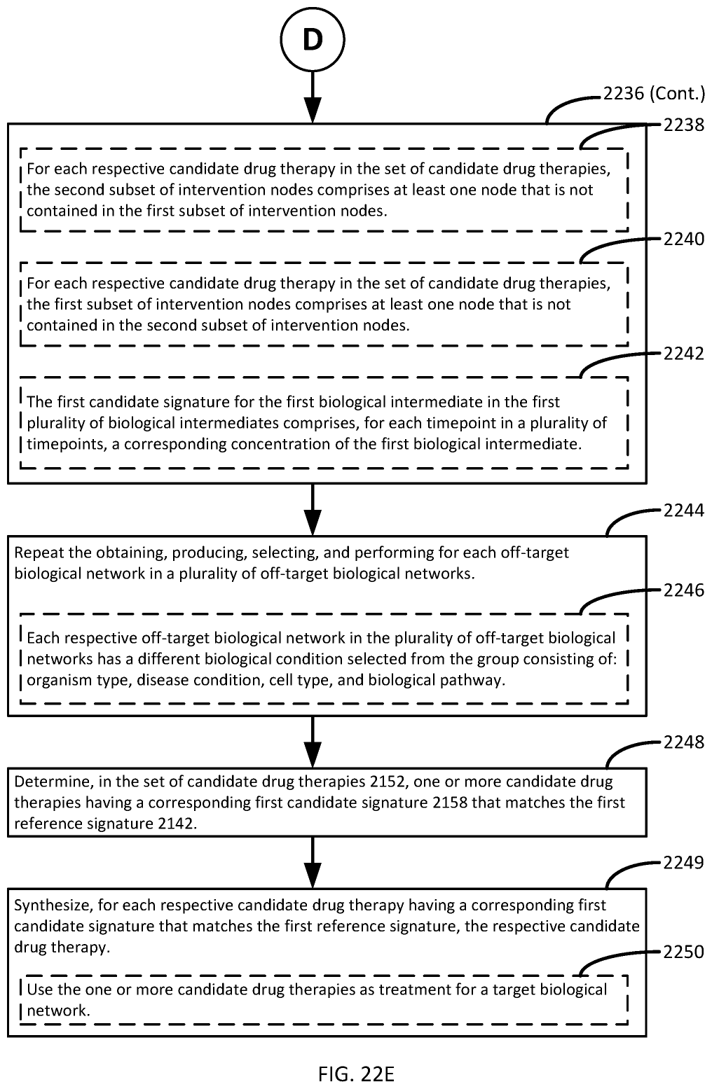

Now that details of a system 100 for determining an effect of a candidate drug therapy have been disclosed, details regarding a flow chart of processes and features, that optionally use the system 100, in accordance with an embodiment of the present disclosure, are disclosed with reference to FIGS. 22A-E. In some embodiments, such processes and features are conducted by the system 100 illustrated in FIGS. 21A-C. In some embodiments, the processes and features referenced in FIGS. 22A-E are conducted without the use of the system illustrated in FIGS. 21A-C.

Referring to Block 2200, FIGS. 22A-E collectively illustrate a method 2200 for determining an effect of a candidate drug therapy 2152. In some embodiments, the method is performed at a computer system comprising one or more processors and a memory.

Advantageously, in some embodiments, the method 200 is used to evaluate one or more off-target effects (e.g., side effects) of a treatment or therapy for a targeted biological network. For instance, in some embodiments, an on-target treatment model (e.g., an existing-treatment model or a new-treatment model for a targeted biological network) characterizes how a particular treatment affects a first biological network (e.g., a target tissue, cell type, pathway, or component thereof), and an off-target treatment model characterizes how that same treatment will affect a particular second biological network (e.g., an off-target tissue, cell type, pathway, or component thereof). In some embodiments, a reference off-target treatment model is used to characterize a reference state for the off-target biological network (e.g., a perturbed or unperturbed state, for instance, in the absence of treatment and/or in the presence of a reference treatment). In some embodiments, the reference state is one free of one or more negative effects or undesired alterations in the off-target biological network. In some implementations, the reference state is represented by, at least, a reference signature in a corresponding reference time-course progression (e.g., a measure of expression, production, and/or activity of one or more components in the off-target biological network).

In some such embodiments, the presently disclosed systems and methods advantageously allow for the determination of candidate drug therapies that induce a similar state in the off-target biological network (e.g., that produce a similar signature and/or time-course progression as the reference signature or time-course progression). Alternatively or additionally, in some embodiments, the presently disclosed systems and methods advantageously allow for the determination of candidate drug therapies that induce a different state in the off-target biological network (e.g., that produce a different signature and/or time-course progression from the reference signature or time-course progression). In some implementations, the presently disclosed systems and methods allow for selection of candidate drug therapies based on similarity to a desired state and/or dissimilarity to an undesired state, as will be apparent to one skilled in the art. In some implementations, one or more candidate drug therapies are selected and/or eliminated from a pool of candidate drug therapies in order to facilitate further drug development and synthesis.

Referring to Block 2202, in some embodiments, the method includes obtaining a reference off-target treatment model 2132 for an off-target biological network 2122. In some embodiments, the off-target biological network 2122 comprises a first plurality of nodes 2124 and a first plurality of biological intermediates 2126, each respective node 2124 in the first plurality of nodes representing a respective biological transition between two or more biological intermediates 2126 in the first plurality of biological intermediates.

In some embodiments, the reference off-target treatment model 2132 comprises a first subset of intervention nodes 2134 and a first subset of control nodes 2144 selected from the first plurality of nodes 2124. In some embodiments, each respective intervention node 2134 in the first subset of intervention nodes comprises (i) a corresponding reference intervention function 2136 for the respective biological transition, responsive to a reference drug therapy, at the respective intervention node 2134, and (ii) a corresponding reference intervention constant 2138, in a set of reference intervention constants, for the respective reference intervention function 2136. In some embodiments, each respective control node 2144 in the first subset of control nodes comprises a corresponding control function 2146 for the respective biological transition, responsive to an absence of the reference drug therapy, at the respective control node 2144.

In some embodiments, the candidate drug therapy is selected from a set of candidate drug therapies for a targeted biological network, other than the off-target biological network. For instance, in some embodiments, a candidate drug therapy is a candidate therapy for treating a first (e.g., targeted) biological network, and the method is used to determine the effect of the candidate drug therapy on a second (e.g., off-target) biological network. Candidate drug therapies and methods of determining the same are described elsewhere herein, for instance, in the sections entitled “Candidate drug therapies” and “Systems and methods for developing an alternative drug therapy,” below.

### Biological Networks

In some embodiments, a biological network (e.g., a targeted biological network and/or an off-target biological network) comprises a corresponding plurality of nodes and a corresponding plurality of biological intermediates, each respective node in the plurality of nodes representing a respective biological transition between two or more biological intermediates in the plurality of biological intermediates.

In some embodiments, a biological network is selected from the group consisting of: organism type (e.g., human, animal, pathogen), disease condition (e.g., diseased, healthy), cell type (e.g., host neuron, host kidney, etc.), and/or biological pathway (e.g., folate pathway, glycolysis pathway, etc.). In some embodiments, the biological pathway is designated as a KEGG pathway.

In some embodiments, a biological network refers to an organism type. In some embodiments, the biological network is selected from the group consisting of: a human (e.g., a male human, female human, fetus, pregnant female, child, or the like), a non-human animal, a plant, a bacterium, a fungus, a virus, or a protist. In some embodiments, the organism type is a pathogen, including but not limited to bacteria, viruses, fungi, protozoa, and/or helminths. In some embodiments, the organism type is a host organism.

In some embodiments, a biological network refers to a disease condition. In some embodiments, the biological network represents a diseased state or a healthy state. In some embodiments, the biological network represents a disease, where the disease is selected from the group consisting of infectious or parasitic diseases; neoplasms; diseases of the blood or blood-forming organs; diseases of the immune system; endocrine, nutritional or metabolic diseases; mental, behavioral or neurodevelopmental disorders; sleep-wake disorders; diseases of the nervous system; diseases of the visual system; diseases of the ear or mastoid process; diseases of the circulatory system; diseases of the respiratory system; diseases of the digestive system; diseases of the skin; diseases of the musculoskeletal system or connective tissue; diseases of the genitourinary system; conditions related to sexual health; diseases related to pregnancy, childbirth or the puerperium; certain conditions originating in the perinatal period; and developmental anomalies.

In some embodiments, the disease is cancer. In some embodiments, the cancer comprises breast cancer, lung cancer, prostate cancer, colorectal cancer, skin cancer, bladder cancer, blood cancer, leukemia, lymphoma, ovarian cancer, pancreatic cancer, kidney cancer, brain cancer, thyroid cancer, stomach (gastric) cancer, liver cancer, cervical cancer, esophageal cancer, bone cancer, a sarcoma, and/or testicular cancer. In some embodiments, the disease is for a stage of cancer, such as stage I, II, III, or IV of the tumor, nodes, and metastasis (TNM) system. In some embodiments, the disease is an infectious disease. In some embodiments, an infectious disease comprises infection by one or more infectious agents, including but not limited to bacteria, viruses, fungi, protozoa, and/or helminths.

In some embodiments, a biological network refers to a cell and/or tissue type. In some embodiments, the biological network is selected from the group consisting of cardiac cells, liver cell, kidney cells, pancreatic cells, neural cells, immune cells, mesenchymal cells, and endothelial cells. In some embodiments, the biological network includes, but is not limited to, cells of the liver, lungs, heart, spleen, pancreas, gastrointestinal tract, kidney, testes, ovaries, brain, reproductive organs, central nervous system, peripheral nervous system, skeletal muscle, endothelium, inner ear, and/or eye. In some embodiments, the biological network includes, but is not limited to, epithelial, connective, muscular, or nervous tissue or cells, and combinations thereof.

In some embodiments, the biological network comprises cells and/or tissue from any eukaryotic (e.g., mammalian) organ system, including but not limited to, for example, from the cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves); reproductive system (ovaries, uterus, mammary glands, testes, vas deferens, seminal vesicles, prostate); respiratory system (pharynx, larynx, trachea, bronchi, lungs, diaphragm); skeletal system (bone, cartilage), and combinations thereof.

In some embodiments, a biological network refers to a biological pathway. In some embodiments, the biological pathway is selected from the group consisting of metabolic pathways, gene regulatory networks, and signal transduction pathways. In some embodiments, the biological pathway is a metabolic pathway selected from the group consisting of Alcohol Metabolism (e.g., Ethanol oxidation, Inositol phosphate metabolism), Carbohydrate and Sugar Metabolism (e.g., Ascorbate and aldarate metabolism, Chondroitin sulfate and dermatan sulfate metabolism, Citric Acid Cycle, Eicosanoid Metabolism, Fructose Metabolism, Galactose Metabolism, Glucose and Energy Metabolism, Glycogen breakdown, Glycolysis and Gluconeogenesis, Glycosaminoglycan degradation, Heparan sulfate biosynthesis and metabolism, Keratan sulfate and keratin metabolism, Mannose Metabolism, Naphthalene metabolism, Pentose phosphate pathway, Pyruvate metabolism, Respiratory Electron Transport, Sorbitol degradation, Starch and sucrose metabolism (Glucoronidation)), Cell Cycle and Mitosis (e.g., Cell Cycle, Mitosis: G1-G1/S phases, Mitosis: G2-G2/M phases, Mitosis: M-M/G1 phases, Mitotic Metaphase and Anaphase), Drug Metabolism (e.g., 4-hydroxybenzoate biosynthesis, Abacavir Metabolism, Acetaminophen metabolism, Butirosin and neomycin biosynthesis, Caffeine Metabolism, Codeine and morphine metabolism, Nicotine metabolism, Tamoxifen metabolism), Lipid and Fatty Acid Metabolism (e.g., Arachidonic acid metabolism, Bile acid and bile salt metabolism, Biosynthesis of unsaturated fatty acids, Cholesterol Biosynthesis, Ether lipid metabolism, Fatty acid beta oxidation, Fatty Acid Biosynthesis, Fatty acid elongation, Fatty acid omega oxidation, Fatty acid, triacylglycerol, and ketone body metabolism, Glycerolipid metabolism, Glycerophospholipid biosynthesis, Glycosphingolipid biosynthesis, Glyoxylate and dicarboxylate metabolism, HETE and HPETE biosynthesis and metabolism, Leukotriene Metabolism, Linoleic acid metabolism, Lipoic acid Metabolism, Metabolism of lipids and lipoproteins, Oleate biosynthesis, Palmitate biosynthesis, Peroxisomal lipid Metabolism, Phospholipid Metabolism, Propanoate metabolism, Prostaglandin Synthesis, Sphingolipid metabolism, Stearate biosynthesis), Neurotransmitter Metabolism (e.g., Acetylcholine Synthesis, Anandamide degradation, Dopamine degradation, Serotonin metabolism), Nucleotide and Nucleoside Metabolism (e.g., Amino sugar and nucleotide sugar metabolism, Purine metabolism, Pyrimidine metabolism, Wybutosine biosynthesis), Peptide Hormone Metabolism (e.g., Catecholamine biosynthesis, Melatonin degradation, Noradrenaline and adrenaline degradation, Peptide hormone metabolism, Regulation of insulin secretion, Renin-angiotensin system), Protein and Amino Acid Metabolism (e.g., Alanine, aspartate and glutamate metabolism, Arginine and proline metabolism, Arylamine Metabolism, Asparagine degradation, beta-Alanine metabolism, Butanoate Metabolism, Citrulline metabolism, Collagen biosynthesis, Creatine metabolism, D-arginine and D-ornithine metabolism, D-Glutamine and D-glutamate metabolism, Glutamine Metabolism, Glutathione metabolism, Glycine, serine, and threonine metabolism, Histamine metabolism, Histidine Metabolism, Lysine Metabolism, Metabolism of proteins, Methionine and cysteine metabolism, One-carbon metabolism, Phenylalanine metabolism, Putrescine degradation, Spermine and spermidine degradation, Taurine and hypotaurine metabolism, tRNA Aminoacylation, Tryptophan Metabolism, Tyrosine metabolism, Urea Cycle, Valine, Leucine and Isoleucine degradation), Steroid Metabolism (e.g., Androgen biosynthesis, Estradiol Metabolism, Estrogen Metabolism, Estrone metabolism, Glucocorticoid and Mineralocorticoid Metabolism, Metabolism of steroid hormones and Vitamin D, Steroid hormone biosynthesis, Thyroid hormone synthesis), Vitamin and Coenzyme Metabolism (e.g., Nicotinate and nicotinamide metabolism, Biotin Metabolism, Folate Metabolism, NAD Metabolism, Pantothenate and CoA biosynthesis, Propionyl-CoA catabolism, Retinoate biosynthesis, Retinol metabolism, Riboflavin Metabolism, Thiamine metabolism, Ubiquinol biosynthesis, Vitamin A and carotenoid metabolism, Vitamin B6 metabolism, Vitamin digestion and absorption), and/or Other Metabolism (e.g., Iron metabolism, Metabolism of nitric oxide, Nitrogen Metabolism, Reversible hydration of carbon dioxide, Selenium Metabolism, Sulfur Metabolism, Benzo(a)pyrene metabolism, Porphyrin Metabolism).

In some embodiments, a biological intermediate in the plurality of biological intermediates is selected from the group consisting of: nucleic acids, including DNA, modified (e.g., methylated) DNA, and/or RNA, including coding (e.g., mRNAs) or non-coding RNA (e.g., sncRNAs); polypeptides or proteins, including post-transcriptionally modified proteins; lipids; carbohydrates; nucleotides (e.g., adenosine triphosphate (ATP), adenosine diphosphate (ADP), and/or adenosine monophosphate (AMP)), including cyclic nucleotides such as cyclic adenosine monophosphate (cAMP) and cyclic guanosine monophosphate (cGMP); metabolites, small molecules such as oxidized and reduced forms of nicotinamide adenine dinucleotide (NADP/NADPH); and/or any combinations thereof.

Alternatively or additionally, in some embodiments, a biological intermediate in the plurality of biological intermediates is an enzyme. In some embodiments, a biological intermediate in the plurality of biological intermediates is a regulatory factor (e.g., a transcription factor). In some embodiments, a biological intermediate in the plurality of biological intermediates is a target of an enzyme and/or a regulatory factor (e.g., a ligand and/or substrate). In some embodiments, a biological intermediate in the plurality of biological intermediates is an outcome of a biological transition induced by an enzyme and/or a regulatory factor (e.g., a product and/or a transcript).

In some embodiments, a biological transition is any interconversion between two or more biological intermediates in a biological network that can be mediated or induced by any biological entity that can be targeted by a treatment, such as a drug therapy. In some embodiments, a biological transition is a chemical interconversion and/or a regulatory function, including but not limited to an inhibition or activation of a process mediated by a biological entity (e.g., a transcription factor, an enzyme, a catalyst, etc.).

In some embodiments, the biological transitions and biological intermediates that are represented in the biological network are dependent on the type of biological network selected. For instance, in some implementations, metabolic pathways comprise proteins or metabolites as biological intermediates, where transitions are chemical interconversions. Alternatively or additionally, in some implementations, gene regulatory networks comprise RNA transcripts as biological intermediates, where transitions represent regulatory functions such as activation or inhibition of expression. Other biological transitions are possible and contemplated for use in the present disclosure, as will be apparent to one skilled in the art.

In some embodiments, a biological transition is between two or more biological intermediates. In some embodiments, a biological transition is between at least 2, at least 3, at least 4, at least 5, or at least 8 biological intermediates. In some embodiments, a biological transition is between no more than 10, no more than 8, no more than 5, no more than 4, or no more than 3 biological intermediates. In some embodiments, a biological transition is between from 2 to 3, from 3 to 5, from 3 to 8, or from 5 to 10 biological intermediates. In some embodiments, a biological transition is between a set of biological intermediates that falls within another range starting no lower than 2 intermediates and ending no higher than 10 intermediates.

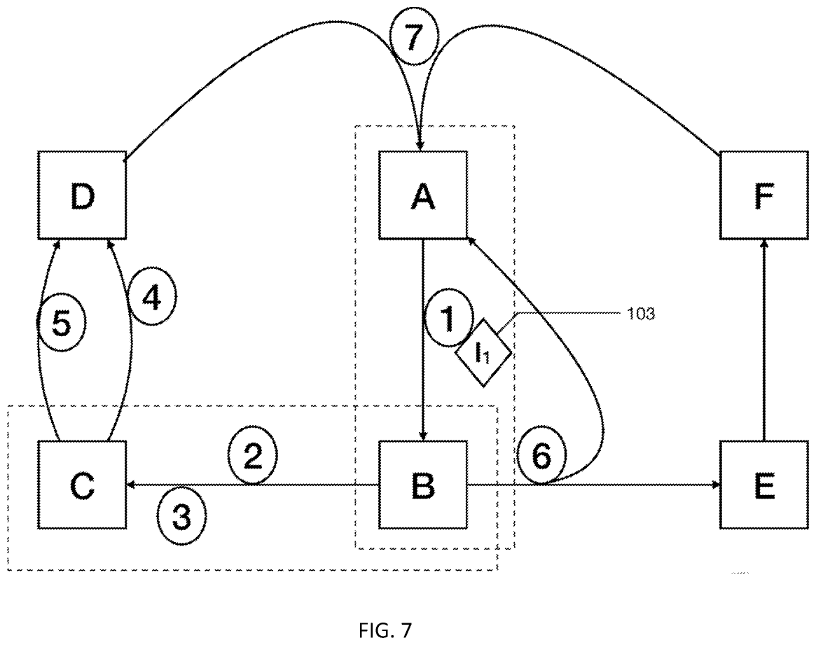

For example, in some embodiments, a biological transition is a chemical interconversion that converts two or more reactants to a product, as illustrated in FIG. 7. Alternatively or additionally, in some embodiments, a biological transition is a chemical interconversion that converts a reactant to two or more products. Any number of reactants can be converted to any number of corresponding products in a respective biological network, as will be apparent to one skilled in the art.

In some embodiments, a biological network is a targeted biological network or an off-target biological network. In some embodiments, a targeted biological network is all or a portion of a pathogen. In some embodiments, a targeted biological network is a disease state (e.g., in a first cell or tissue type). Alternatively or additionally, in some embodiments, an off-target biological network is a biological network, other than the targeted biological network, in which an effect of one or more candidate drug therapies are evaluated (e.g., a host organism and/or a healthy state in a second cell or tissue type).

### Off-Target Biological Network

In some embodiments, the first plurality of biological intermediates comprises at least 3 biological intermediates.

In some embodiments, the first plurality of biological intermediates comprises at least 2, at least 3, at least 4, at least 5, at least 6, at least 7, at least 8, at least 10, at least 15, at least 20, at least 30, or at least 50 biological intermediates. In some embodiments, the first plurality of biological intermediates comprises no more than 100, no more than 50, no more than 30, no more than 20, no more than no more than 10, no more than 8, no more than 5, no more than 4, or no more than 3 biological intermediates. In some embodiments, the first plurality of biological intermediates consists of from 2 to 5, from 2 to 10, from 4 to 15, from 10 to 25, from 12 to 40, from 20 to 60, or from 50 to 100 biological intermediates. In some embodiments, the first plurality of biological intermediates comprises falls within another range starting no lower than 2 intermediates and ending no higher than 100 intermediates.

Referring to Block 2204, in some embodiments, each respective biological intermediate in the first plurality of biological intermediates is a polypeptide or a nucleic acid. In some embodiments, each respective biological intermediate in the first plurality of biological intermediates is selected from any of the biological intermediates disclosed elsewhere herein (see, e.g., the section entitled “Biological networks,” above).

In some embodiments, the first plurality of nodes comprises at least 5 nodes.

In some embodiments, the first plurality of nodes comprises at least 2, at least 3, at least 4, at least 5, at least 6, at least 7, at least 8, at least 10, at least 15, at least 20, at least 30, or at least 50 nodes. In some embodiments, the first plurality of nodes comprises no more than 100, no more than 50, no more than 30, no more than 20, no more than no more than 10, no more than 8, no more than 5, no more than 4, or no more than 3 nodes. In some embodiments, the first plurality of nodes consists of from 2 to 5, from 2 to 10, from 4 to 15, from 10 to 25, from 12 to 40, from 20 to 60, or from 50 to 100 nodes. In some embodiments, the first plurality of nodes comprises falls within another range starting no lower than 2 nodes and ending no higher than 100 nodes.

Referring to Block 2206, in some embodiments, for a respective node in the first plurality of nodes, the respective biological transition is a chemical interconversion or a regulatory function. In some embodiments, a respective biological transition is selected from any of the biological transitions disclosed elsewhere herein (see, e.g., the section entitled “Biological networks,” above). Referring to Block 2208, in some embodiments, the off-target biological network is selected from the group consisting of: an organism type, a disease condition, a cell type, and a biological pathway. In some embodiments, the off-target biological network is selected from any of the biological networks disclosed elsewhere herein (see, e.g., the section entitled “Biological networks,” above).

In some embodiments, the method further comprises determining a targeted biological network, other than the off-target biological network.

Referring to Block 2210, in some embodiments, the reference drug therapy is selected as a treatment for a targeted biological network. Any suitable targeted biological networks and/or any suitable off-target biological network other than the targeted biological network is contemplated for use in the present disclosure, as will be apparent to one skilled in the art. In some embodiments, the selection of a targeted biological network and/or a corresponding off-target biological network depends on the focus of the study or approach being performed. For instance, it may be desirable to assess whether a drug therapy for a first cell or tissue type has off-target effects in a second cell or tissue type, and thereby minimize or reduce any adverse effects upon exposure to the drug therapy. In some embodiments, the targeted biological network is selected from any of the biological networks disclosed elsewhere herein (see, for example, the section entitled “Biological networks,” above).

Referring to Block 2212, in some embodiments, the targeted biological network is for a first organism, and the off-target biological network is for a second organism. In some embodiments, the first organism is a pathogen, and the second organism is a host organism. In some embodiments, the pathogen is E. coli. In some embodiments, the pathogen is any of the pathogens or infectious agents disclosed elsewhere herein (see, e.g., the section entitled “Biological networks,” above). In some embodiments, the host organism is a human.

Alternatively or additionally, referring to Block 2214, in some embodiments, the targeted biological network is a disease cell and the off-target biological network is a healthy cell. In some embodiments, the targeted biological network is a cancer cell and the off-target biological network is a normal (e.g., non-cancerous) cell.

Alternatively or additionally, in some embodiments, the targeted biological network is a first cell type (e.g., a neuron) and the off-target biological network is a second cell type (e.g., a kidney cell).

Alternatively or additionally, referring to Block 2216, in some embodiments, the targeted biological network is a first biological pathway and the off-target biological network is a second biological pathway, other than the first biological pathway. In some embodiments, the targeted biological network is a folate pathway and the off-target biological network is another biological pathway other than the folate pathway.

### Plurality of Networks

In some embodiments, the method is repeated for each off-target biological network in a plurality of off-target biological networks. Referring to Block 2234, in some embodiments, the method further includes repeating the obtaining, producing, selecting, and performing for each off-target biological network in a plurality of off-target biological networks.

In some embodiments, the plurality of off-target biological networks includes at least 2, at least 3, at least 4, at least 5, at least 6, at least 7, at least 8, at least 10, at least 15, at least 20, or at least 30 biological networks. In some embodiments, the plurality of off-target biological networks comprises no more than 50, no more than 30, no more than 20, no more than no more than 10, no more than 8, no more than 5, no more than 4, or no more than 3 biological networks. In some embodiments, the plurality of off-target biological networks consists of from 2 to 5, from 2 to 10, from 4 to 15, from 10 to 25, from 12 to 40, or from 20 to 50 biological networks. In some embodiments, the plurality of off-target biological networks comprises falls within another range starting no lower than 2 biological networks and ending no higher than 50 biological networks.

In some embodiments, each respective off-target biological network in the plurality of off-target biological networks is a different biological network. Referring to Block 2236, in some embodiments, each respective off-target biological network in the plurality of off-target biological networks is a different biological network selected from the group consisting of: organism type, disease condition, cell type, and biological pathway.

In some embodiments, the method further includes obtaining a different reference off-target model for each off-target biological network of interest. For instance, in some embodiments, a first cell type (e.g., alveolar cells) exhibits a different signature and/or time-course progression upon exposure to a presence or absence of a drug therapy (e.g., a reference drug therapy and/or a candidate drug therapy), relative to a second cell type (e.g., gut cells, neuron cells, etc.). In some embodiments, the method includes comparing a response for a candidate drug therapy relative to a response for a reference drug therapy, where each of the response for a candidate drug therapy and the response for a reference drug therapy are specific to the type biological network undergoing evaluation.

### Reference Drug Therapies

In some embodiments, as noted above, the reference drug therapy is used for treating a first (e.g., targeted) biological network.

In some embodiments, the reference drug therapy is a drug therapy that has a first effect on the targeted biological network, and a second effect on one or more off-target biological networks. In some embodiments, the first effect is the same or different from the second effect.

In some embodiments, the reference drug therapy is selected from a compound database and/or library. For example, in some embodiments, the reference drug therapy is selected from a library of known inhibitors, chemical, small molecules, and/or agonists, among others.

In some embodiments, the reference drug therapy is determined by generating, for the off-target biological network, an existing-treatment mathematical model and/or a new-treatment mathematical model, as described elsewhere herein (see, e.g., the section entitled “Systems and methods for developing an alternative drug therapy,” below).

In some embodiments, the reference drug therapy is selected from the group consisting of: polypeptides, proteins, RNA, DNA, lipids, small molecules, RNAi, chemicals, and/or antibodies.

### Obtaining Reference Off-Target Treatment Model

In some embodiments, the method includes determining values for the reference off-target treatment model empirically and/or experimentally. In some embodiments, values for the reference off-target treatment model are obtained from an existing data source (e.g., from a database and/or a repository). For instance, in some embodiments, non-limiting examples of data sources for values include protein kinetics and/or gene expression repositories storing differential expression and/or intermediate production responsive to drug treatment and/or chemical exposure.

In some embodiments, the method includes generating the reference off-target treatment model from empirically derived or previously obtained values. In some embodiments, an existing reference off-target treatment model is used. In some embodiments, the reference off-target treatment model is obtained by any suitable means, including generating the model de novo or using a previously generated model, as will be apparent to one skilled in the art.

In some embodiments, reference off-target treatment model is obtained by generating, for the off-target biological network, an existing-treatment mathematical model and/or a new-treatment mathematical model, where the existing treatment and/or the new treatment is a treatment for a targeted biological network. For instance, in some embodiments, the method further includes selecting, as a basis for determining the reference off-target treatment model, an existing or new treatment for a targeted biological network that also has a particular or desired effect on an off-target biological network.

In some embodiments, the reference off-target treatment model characterizes a “baseline” for the off-target biological network; for instance, in some implementations, it models how the network behaves in the absence of treatment and/or any side effects. In some embodiments, the reference off-target treatment model characterizes a desired status or outcome for the off-target biological network, such as modeling a desired state or behavior of the off-target biological network.

In some embodiments, a respective treatment model (e.g., a reference treatment model and/or a candidate treatment model) comprises a kinetics model. In some embodiments, a respective treatment model comprises a differential equation model. In some embodiments, a respective treatment model comprises a stochastic model, such as a stochastic differential equation. In some embodiments, a respective treatment model comprises a rate equation, an Arrhenius equation, a Michaelis-Menten equation, and/or a Hill equation.

A rate equation is a mathematical expression that describes the relationship between the rate of a chemical reaction and the concentration of its reactants. The Arrhenius equation is a formula for the temperature dependence of reaction rates. The Hill equation refers to two closely related equations that reflect the binding of ligands to macromolecules, as a function of the ligand concentration. A Michaelis-Menten equation is an equation relating reaction velocity to substrate concentration for a system where a substrate S binds reversibly to an enzyme E to form an enzyme-substrate complex ES, which then reacts irreversibly to generate a product P and to regenerate the free enzyme E.

In some embodiments, the reference off-target treatment model comprises a Michaelis-Menten equation.

### Intervention Functions and Constants

In some embodiments, the reference off-target treatment model comprises a first subset of intervention nodes and a first subset of control nodes selected from the first plurality of nodes, each respective intervention node in the first subset of intervention nodes comprises (i) a corresponding reference intervention function for the respective biological transition, responsive to a reference drug therapy, at the respective intervention node, and (ii) a corresponding reference intervention constant, in a set of reference intervention constants, for the respective reference intervention function, and each respective control node in the first subset of control nodes comprises a corresponding control function for the respective biological transition, responsive to an absence of the reference drug therapy, at the respective control node.

In some embodiments, an intervention function models a respective biological transition responsive to a corresponding treatment. For instance, in some embodiments, an intervention function (e.g., a reference intervention function and/or a candidate intervention function) models an enzymatic activity or change thereof, a change in concentration, or other biological transition, that is induced at a respective node upon exposure to or treatment with a corresponding drug therapy (e.g., a reference drug therapy and/or a candidate drug therapy).

In some embodiments, an intervention function is an inhibition function or an acceleration function. In some embodiments, an intervention function refers to a decrease and/or inhibition of activity, including but not limited to enzyme inhibition, transcriptional inhibition, gene silencing, knockdown, and/or competitive inhibition. In some embodiments, an intervention function refers to an increase and/or activation of activity, including but not limited to enzyme activation, transcriptional activation, gene promoter activity, overexpression, and/or ligand or agonist-mediated activity.

In some embodiments, an intervention constant is a value that indicates a rate associated with the intervention function, including, but not limited to, a rate of inhibition or acceleration, and/or a rate of activity for the biological transition represented by the respective node. In some embodiments, an intervention constant is a value that represents the effect on the speed of the biological transition induced by a corresponding treatment (e.g., a reference drug therapy and/or a candidate drug therapy).

In some embodiments, an intervention constant is a rate constant or a node velocity that represents a rate at which a biological transition occurs (e.g., an enzyme converting one or more reactants to one or more products). In some embodiments, an intervention constant is a concentration constant that represents a concentration of one or more biological intermediates that are consumed by and/or produced by a respective biological transition.

In some embodiments, an intervention constant is a parameter for a respective treatment model (e.g., a reference off-target treatment model, a candidate off-target treatment model, etc.). In some embodiments, an intervention constant is an inhibition constant that represents a slowing, deceleration, or substantial stopping of a biological transition at a respective node. Alternatively or additionally, in some embodiments, an intervention constant is an acceleration constant that represents a starting, activation, acceleration, or speeding up of a biological transition at a respective node.

Referring to Block 2218, in some embodiments, for a respective intervention node (alternatively herein, “intervened-upon” nodes) in the first subset of intervention nodes, the corresponding reference intervention function is an inhibition function. Referring to Block 2220, in some embodiments, for a respective intervention node in the first subset of intervention nodes, the corresponding reference intervention constant is an inhibition constant. In some embodiments, for each respective intervention node in the first subset of intervention nodes, the corresponding reference intervention function is an inhibition function and the corresponding reference intervention constant is an inhibition constant.

Referring to Block 2222, in some embodiments, for a respective intervention node in the first subset of intervention nodes, the corresponding reference intervention function is an acceleration function. Referring to Block 2224, in some embodiments, for a respective intervention node in the first subset of intervention nodes, the corresponding reference intervention constant is an acceleration constant. In some embodiments, for each respective intervention node in the first subset of intervention nodes, the corresponding reference intervention function is an acceleration function and the corresponding reference intervention constant is an acceleration constant.

In some embodiments, for each respective intervention node in the first subset of intervention nodes, the corresponding reference intervention function comprises any of the models, equations, or functions disclosed elsewhere herein (see, e.g., the section entitled “Obtaining reference off-target treatment model,” above).

Referring to Block 2226, in some embodiments, for each respective intervention node in the first subset of intervention nodes, the corresponding reference intervention function is a Michaelis-Menten equation. Referring to Block 2228, in some embodiments, the first subset of intervention nodes (e.g., for the reference off-target treatment model) comprises at least 1 intervention node. In some embodiments, the first subset of intervention nodes comprises at least 2 intervention nodes.

In some embodiments, the first subset of intervention nodes includes at least 2, at least 3, at least 4, at least 5, at least 6, at least 7, at least 8, at least 10, at least 15, at least 20, or at least 30 nodes. In some embodiments, the first subset of intervention nodes comprises no more than 50, no more than 30, no more than 20, no more than no more than 10, no more than 8, no more than 5, no more than 4, or no more than 3 nodes. In some embodiments, the first subset of intervention nodes consists of from 2 to 5, from 2 to 10, from 4 to 15, from 10 to 25, from 12 to 40, or from 20 to 50 nodes. In some embodiments, the first subset of intervention nodes comprises falls within another range starting no lower than 2 nodes and ending no higher than 50 nodes.

Referring to Block 2230, in some embodiments, the first subset of intervention nodes consists of only one node.

In some embodiments, the set of reference intervention constants comprises at least 2, at least 3, at least 4, at least 5, at least 6, at least 7, at least 8, at least 10, at least 15, at least 20, at least 30, or at least 50 intervention constants. In some embodiments, the set of reference intervention constants comprises no more than 100, no more than 50, no more than 30, no more than 20, no more than no more than 10, no more than 8, no more than 5, no more than 4, or no more than 3 intervention constants. In some embodiments, the set of reference intervention constants consists of from 2 to 5, from 2 to 10, from 4 to 15, from 10 to 25, from 12 to 40, from 20 to 60, or from 50 to 100 intervention constants. In some embodiments, the set of reference intervention constants comprises falls within another range starting no lower than 2 intervention constants and ending no higher than 100 intervention constants.

In some embodiments, the set of reference intervention constants comprises, for each respective intervention node in the first subset of intervention nodes, a corresponding one or more intervention constants for the corresponding reference intervention function (e.g., each function is associated with at least one constant). In some embodiments, a reference intervention function comprises one or more reference intervention constants. In some embodiments, a reference intervention function comprises a plurality of reference intervention constants (e.g., a function can be associated with more than one constant).

In some embodiments, a corresponding intervention function (e.g., a reference and/or candidate intervention function) for a respective intervention node represents one or more biological transitions responsive to a drug therapy (e.g., a reference and/or candidate drug therapy). In some embodiments, a corresponding intervention function (e.g., a reference and/or candidate intervention function) for a respective intervention node represents a plurality of biological transitions responsive to a drug therapy (e.g., a reference and/or candidate drug therapy). In some embodiments, the plurality of biological transitions comprises at least 2, at least 3, or at least 4 biological transitions. In some embodiments, the plurality of biological transitions comprises no more than 5, no more than 4, or no more than 3 biological transitions. In some embodiments, the plurality of biological transitions consists of from 2 to 5 biological transitions.

### Control Functions and Constants

In some embodiments, a control function models a respective biological transition responsive to an absence of treatment. For instance, in some embodiments, a control function (e.g., a control function for a respective control node in a first subset of control nodes for a reference off-target treatment model, and/or a control function for a respective control node in a second subset of control nodes for a candidate off-target treatment model) models an enzymatic activity or change thereof, a change in concentration, or other biological transition, that occurs at a respective node in the absence of any drug therapy (e.g., a baseline activity).

As described above, a control function, in some embodiments, includes but is not limited to any decrease, inhibition, increase, and/or activation of activity, including but not limited to enzyme inhibition, transcriptional inhibition, gene silencing, knockdown, competitive inhibition, enzyme activation, transcriptional activation, gene promoter activity, overexpression, and/or ligand or agonist-mediated activity. In some embodiments, a control function is selected from any of the intervention functions disclosed elsewhere herein.

In some embodiments, a respective control node in the first subset of control nodes further comprises a corresponding control constant, in a set of control constants.

As described above, in some embodiments, a control constant is a value that indicates a rate associated with a control function, including, but not limited to, a rate of inhibition or acceleration, and/or a rate of activity for the biological transition represented by the respective control node. In some embodiments, a control constant is selected from any of the intervention constants disclosed elsewhere herein.

In some embodiments, for each respective control node in the first subset of control nodes, the corresponding control function comprises any of the models, equations, or functions disclosed elsewhere herein (see, e.g., the section entitled “Obtaining reference off-target treatment model,” above).

In some embodiments, for each respective control node in the first subset of control nodes, the corresponding control function is a Michaelis-Menten equation.

In some embodiments, the first subset of control nodes (e.g., for the reference off-target treatment model) comprises at least 1 control node. In some embodiments, the first subset of control nodes comprises at least 2 control nodes.

In some embodiments, the first subset of control nodes includes at least 2, at least 3, at least 4, at least 5, at least 6, at least 7, at least 8, at least 10, at least 15, at least 20, or at least 30 nodes. In some embodiments, the first subset of control nodes comprises no more than 50, no more than 30, no more than 20, no more than no more than 10, no more than 8, no more than 5, no more than 4, or no more than 3 nodes. In some embodiments, the first subset of control nodes consists of from 2 to 5, from 2 to 10, from 4 to 15, from 10 to 25, from 12 to 40, or from 20 to 50 nodes. In some embodiments, the first subset of control nodes comprises falls within another range starting no lower than 2 nodes and ending no higher than 50 nodes.

In some embodiments, the reference off-target treatment model comprises a plurality of nodes that consists of the first subset of intervention nodes and the first subset of control nodes. In other words, in some embodiments, each node in the plurality of nodes for the reference off-target treatment model, other than the first subset of intervention nodes, is a control node.

In some embodiments, the set of control constants comprises at least 2, at least 3, at least 4, at least 5, at least 6, at least 7, at least 8, at least 10, at least 15, at least 20, at least 30, or at least 50 control constants. In some embodiments, the set of control constants comprises no more than 100, no more than 50, no more than 30, no more than 20, no more than no more than 10, no more than 8, no more than 5, no more than 4, or no more than 3 control constants. In some embodiments, the set of control constants consists of from 2 to 5, from 2 to 10, from 4 to 15, from 10 to 25, from 12 to 40, from 20 to 60, or from 50 to 100 control constants. In some embodiments, the set of control constants comprises falls within another range starting no lower than 2 control constants and ending no higher than 100 control constants.

In some embodiments, the set of control constants comprises, for each respective control node in the first subset of control nodes, a corresponding one or more control constants for the corresponding control function (e.g., each function is associated with at least one constant). In some embodiments, a control function comprises one or more control constants. In some embodiments, a control function comprises a plurality of control constants (e.g., a function can be associated with more than one constant).

In some embodiments, a corresponding control function for a respective control node represents one or more biological transitions responsive to an absence of drug therapy. In some embodiments, a corresponding control function for a respective control node represents a plurality of biological transitions responsive to an absence of drug therapy. In some embodiments, the plurality of biological transitions comprises at least 2, at least 3, or at least 4 biological transitions. In some embodiments, the plurality of biological transitions comprises no more than 5, no more than 4, or no more than 3 biological transitions. In some embodiments, the plurality of biological transitions consists of from 2 to 5 biological transitions.

### Reference Time-Course Progression and Reference Signatures

Referring to Block 2231, in some embodiments, the method further includes producing, using the reference off-target treatment model 2132, a corresponding reference time-course progression 2140 for the off-target biological network 2122 comprising at least a first reference signature 2142 for a first biological intermediate 2126 in the first plurality of biological intermediates.

In some embodiments, the reference time-course progression comprises one or more reference signatures. In some embodiments, the reference time-course progression comprises a plurality of reference signatures.

In some embodiments, the plurality of reference signatures includes at least 2, at least 3, at least 4, at least 5, at least 6, at least 7, at least 8, at least 10, at least 15, at least 20, or at least 30 signatures. In some embodiments, the plurality of reference signatures comprises no more than 50, no more than 30, no more than 20, no more than no more than 10, no more than 8, no more than 5, no more than 4, or no more than 3 signatures. In some embodiments, the plurality of reference signatures consists of from 2 to 5, from 2 to 10, from 4 to 15, from 10 to 25, from 12 to 40, or from 20 to 50 signatures. In some embodiments, the plurality of reference signatures comprises falls within another range starting no lower than 2 signatures and ending no higher than 50 signatures.

Referring to Block 2232, in some embodiments, the first reference signature for the first biological intermediate in the first plurality of biological intermediates comprises, for each timepoint in a plurality of timepoints, a corresponding concentration of the first biological intermediate.

In some embodiments, a reference signature is a measure of activity, amount, or change thereof, for a respective biological intermediate in the first plurality of biological intermediates for the respective off-target biological network.

In some embodiments, a reference signature is an amount of a biological intermediate, including but not limited to a concentration, an absolute quantity, a detectable label, a fold change, a cycle threshold, or any representation thereof. For example, a detectable label includes, but is not limited to, a measurable amount of a fluorophore or an immunolabel. The amount of a biological intermediate can be measured using any means known in the art, including but not limited to polymerase chain reaction (PCR), quantitative PCR, antibody-based detection, western blot, southern blot, fluorescent imaging, cell sorting, flow cytometry, gel electrophoresis, mass spectrometry, enzyme activity assay, colorimetric assay, and/or chemiluminescent assay. In some embodiments, a reference signature comprises a concentration of a biological intermediate. In some embodiments, a reference signature is an amount of a biological intermediate that reaches a given threshold value.

In some embodiments, a reference time-course progression comprises a sequence of events. For example, the sequence of events can be defined at least in part by a first biological intermediate having a first concentration that satisfies a first threshold followed by a second biological intermediate having a second concentration that satisfies a second threshold. As another example, the sequence of events is defined at least in part by a first biological intermediate having a first concentration that satisfies a first threshold followed by the first biological intermediate having a second concentration that satisfies a second threshold. In another embodiment, the time-course progression comprises a first concentration of a first biological intermediate that satisfies a first threshold and a second concentration of a second biological intermediate that satisfies a second threshold. In another embodiment, the time-course progression comprises concentrations of one or more biological intermediates in a set of biological intermediates entering a range such that the concentrations together do not deviate from a set of target concentrations for each of the biological intermediates by equal to or more than a deviation threshold. In some such embodiments, the deviation between the set of concentrations and the set of target concentrations is determined using a root-mean squared calculation.

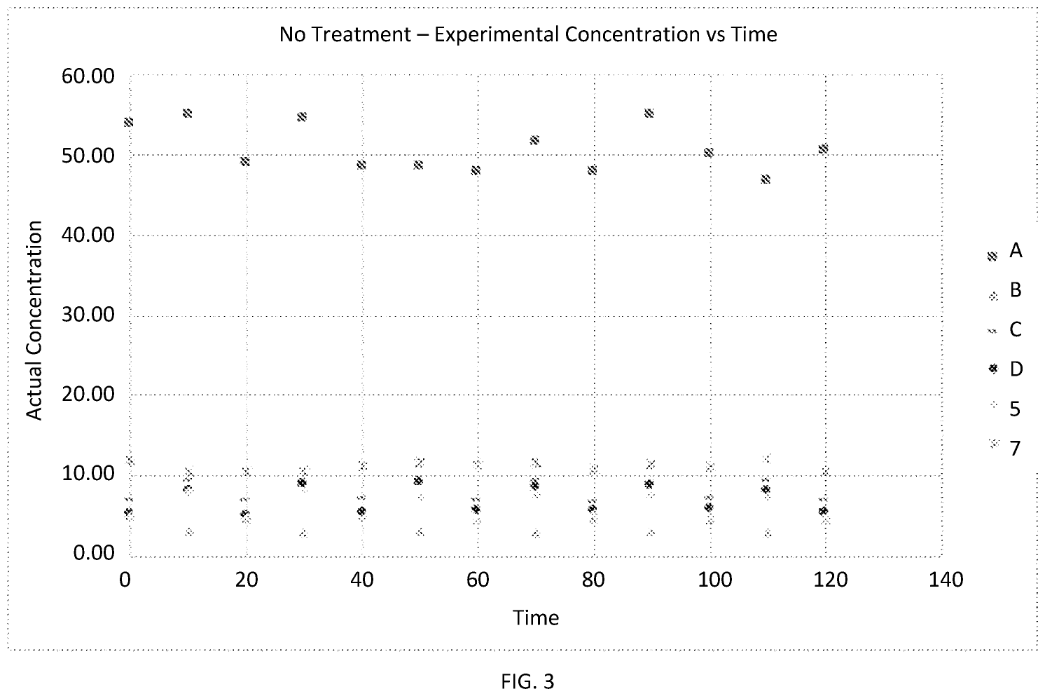

In some embodiments, the reference time-course progression comprises a plurality of timepoints, where the reference signature represents a plurality of events (e.g., measurements or values) for the first biological intermediate. In some embodiments, the reference signature comprises, at each respective timepoint in the plurality of timepoints, a corresponding event in the plurality of events (e.g., a corresponding measurement or value for the first biological intermediate at the respective timepoint). For example, in some embodiments, the reference time-course progression comprises experimental concentration over time, as illustrated in FIGS. 3, 6, 13, and 20.

In some embodiments, the plurality of timepoints comprises at least 2, at least 3, at least 4, at least 5, at least 10, at least 15, at least 20, at least 30, at least 50, at least 100, at least 200, at least 300, or at least 500 timepoints. In some embodiments, the plurality of timepoints comprises no more than 1000, no more than 500, no more than 300, no more than 100, no more than 50, no more than 30, no more than 20, no more than no more than 10, no more than 8, no more than 5, no more than 4, or no more than 3 timepoints. In some embodiments, the plurality of timepoints consists of from 2 to 5, from 2 to 10, from 8 to 30, from 20 to 80, from 50 to 200, from 100 to 500, or from 500 to 1000 timepoints. In some embodiments, the plurality of timepoints comprises falls within another range starting no lower than 2 timepoints and ending no higher than 1000 timepoints.

In some embodiments, the plurality of timepoints comprises a duration of at least 1 second, at least 5 seconds, at least 30 seconds, at least 1 minute, at least 5 minutes, at least 10 minutes, at least 1 hour, at least 12 hours, at least 24 hours, or at least 48 hours. In some embodiments, the plurality of timepoints comprises a duration of no more than 72 hours, no more than 48 hours, no more than 24 hours, no more than 12 hours, no more than 1 hour, no more than 10 minutes, no more than 5 minutes, no more than 1 minute, no more than 30 second, or no more than 5 seconds. In some embodiments, the plurality of timepoints comprises a duration of from 1 second to 1 minute, from 1 minute to 10 minutes, from 10 minutes to 1 hour, from 1 hour to 12 hours, from 12 hours to 24 hours, or from 24 hours to 72 hours. In some embodiments, the plurality of timepoints comprises a duration that falls within another range starting no lower than 1 second and ending no higher than 72 hours.

In some embodiments, the reference time-course progression comprises a plot of enzyme kinetics for at least one enzyme or biological intermediate thereof.

### Candidate Drug Therapies

In some embodiments, the method further includes selecting a set of candidate drug therapies.

In some embodiments, the set of candidate drug therapies comprises at least 2, at least 3, at least 4, at least 5, at least 10, at least 15, at least 20, at least 30, at least 50, at least 100, at least 200, at least 300, or at least 500 candidate drug therapies. In some embodiments, the set of drug therapies comprises no more than 1000, no more than 500, no more than 300, no more than 100, no more than 50, no more than 30, no more than 20, no more than no more than 10, no more than 8, no more than 5, no more than 4, or no more than 3 drug therapies. In some embodiments, the set of candidate drug therapies consists of from 2 to 5, from 2 to 10, from 8 to 30, from 20 to 80, from 50 to 200, from 100 to 500, or from 500 to 1000 drug therapies. In some embodiments, the set of candidate drug therapies comprises falls within another range starting no lower than 2 drug therapies and ending no higher than 1000 drug therapies.

In some embodiments, the set of candidate drug therapies is a plurality of candidate drug therapies. Referring to Block 2234, in some embodiments, the method further includes selecting a plurality of candidate drug therapies 2152. In some embodiments, each candidate drug therapy in the set of candidate drug therapies is a candidate drug therapy for a targeted biological network.

In some embodiments, a respective candidate drug therapy in the set of candidate drug therapies is selected from a compound database and/or library. In some embodiments, the reference drug therapy is selected from a library of known inhibitors, chemical, small molecules, and/or agonists, among others. In some embodiments, a respective candidate drug therapy in the set of candidate drug therapies is selected from the group consisting of: polypeptides, proteins, RNA, DNA, lipids, small molecules, RNAi, chemicals, and/or antibodies. In some embodiments, a respective candidate drug therapy comprises any of the therapies disclosed elsewhere herein, for instance, in the section entitled “Reference drug therapies,” above.

In some embodiments, a candidate drug therapy is determined by generating, for a targeted biological network, a new-treatment mathematical model, as described elsewhere herein (see, e.g., the section entitled “Systems and methods for developing an alternative drug therapy,” below). In some embodiments, a candidate drug therapy corresponds to a respective new-treatment mathematical model in a plurality of new-treatment mathematical models for a targeted biological network. For instance, in some embodiments, each candidate drug therapy is a candidate drug therapy for a targeted biological network, and the method further includes assessing whether each candidate drug therapy has a particular effects on an off-target biological network as characterized by one or more candidate signatures.

### Determining Candidate Off-Target Treatment Models

Referring to Block 2236, in some embodiments, the method further includes performing, for each respective candidate drug therapy 2152 in the set (e.g., plurality) of candidate drug therapies, a procedure comprising determining a respective candidate off-target treatment model 2154 for the off-target biological network 2122. In some embodiments, the respective candidate off-target treatment model 2154 comprises a second subset of intervention nodes 2134 and a second subset of control nodes 2144 selected from the first plurality of nodes 2124. In some embodiments, each respective intervention node 2134 in the second subset of intervention nodes comprises (i) a corresponding candidate intervention function 2136 for the respective biological transition, responsive to the respective candidate drug therapy, at the respective intervention node 2134, and (ii) a corresponding candidate intervention constant 2138, in a set of candidate intervention constants, for the respective candidate intervention function 2136. In some embodiments, each respective control node 2144 in the second subset of control nodes comprises a corresponding control function 2146 for the respective biological transition, responsive to an absence of the respective candidate drug therapy, at the respective control node 2144.

In some embodiments, the procedure further comprises generating, using the respective candidate off-target treatment model 2154, a corresponding candidate time-course progression 2156 for the off-target biological network 2122 comprising at least a first candidate signature 2158 for the first biological intermediate 2126 in the first plurality of biological intermediates.

In some embodiments, for each respective candidate drug therapy in the set (e.g., plurality) of candidate drug therapies, the second subset of intervention nodes (e.g., for the candidate off-target treatment model) comprises at least 1 intervention node. In some embodiments, the second subset of intervention nodes comprises at least 2 intervention nodes.

In some embodiments, the second subset of intervention nodes includes at least 2, at least 3, at least 4, at least 5, at least 6, at least 7, at least 8, at least 10, at least 15, at least 20, or at least 30 nodes. In some embodiments, the second subset of intervention nodes comprises no more than 50, no more than 30, no more than 20, no more than no more than 10, no more than 8, no more than 5, no more than 4, or no more than 3 nodes. In some embodiments, the second subset of intervention nodes consists of from 2 to 5, from 2 to 10, from 4 to 15, from 10 to 25, from 12 to 40, or from 20 to 50 nodes. In some embodiments, the second subset of intervention nodes comprises falls within another range starting no lower than 2 nodes and ending no higher than 50 nodes.

Referring to Block 2238, in some embodiments, for each respective candidate drug therapy in the set (e.g., the plurality) of candidate drug therapies, the second subset of intervention nodes comprises at least one node that is not contained in the first subset of intervention nodes. For example, in some embodiments, the candidate therapy targets one or more nodes (e.g., biological targets) that are not targeted by the reference therapy.

Referring to Block 2240, in some embodiments, for each respective candidate drug therapy in the set (e.g., the plurality) of candidate drug therapies, the first subset of intervention nodes comprises at least one node that is not contained in the second subset of intervention nodes. For example, in some embodiments, the candidate therapy does not directly target at least one node (e.g., biological target) that is targeted by the reference therapy.

In some embodiments, one or more nodes are common to both the first subset of intervention nodes and the second subset of intervention nodes. In some embodiments, the first subset of intervention nodes and the second subset of intervention nodes do not share any nodes. For example, in some embodiments, the candidate treatment does not target any nodes (e.g., biological targets) that are also targeted by the reference treatment. In other words, in some embodiments, the candidate treatment targets alternative nodes, for instance, where targets of the reference treatment are resistant to treatment, or where targets of the reference treatment have known adverse effects.

In some embodiments, the set of candidate intervention constants comprises at least 2, at least 3, at least 4, at least 5, at least 6, at least 7, at least 8, at least 10, at least 15, at least 20, at least 30, or at least 50 intervention constants. In some embodiments, the set of candidate intervention constants comprises no more than 100, no more than 50, no more than 30, no more than 20, no more than no more than 10, no more than 8, no more than 5, no more than 4, or no more than 3 intervention constants. In some embodiments, the set of candidate intervention constants consists of from 2 to 5, from 2 to 10, from 4 to 15, from 10 to 25, from 12 to 40, from 20 to 60, or from 50 to 100 intervention constants. In some embodiments, the set of candidate intervention constants comprises falls within another range starting no lower than 2 intervention constants and ending no higher than 100 intervention constants.

In some embodiments, the set of candidate intervention constants comprises, for each respective intervention node in the second subset of intervention nodes, a corresponding one or more candidate intervention constants for the corresponding candidate intervention function (e.g., each function is associated with at least one constant). In some embodiments, a candidate intervention function comprises one or more candidate intervention constants. In some embodiments, a candidate intervention function comprises a plurality of candidate intervention constants (e.g., a function can be associated with more than one constant).

In some embodiments, a respective candidate intervention function is selected from any of the intervention functions disclosed elsewhere herein (see, e.g., the section entitled “Obtaining reference off-target treatment model,” above). In some embodiments, a respective candidate intervention constant is selected from any of the intervention constants disclosed elsewhere herein (see, e.g., the section entitled “Obtaining reference off-target treatment model,” above).

In some embodiments, for each respective candidate drug therapy in the set (e.g., plurality) of candidate drug therapies, the second subset of control nodes (e.g., for the candidate off-target treatment model) comprises at least 1 control node. In some embodiments, the second subset of control nodes comprises at least 2 control nodes.

In some embodiments, the second subset of control nodes includes at least 2, at least 3, at least 4, at least 5, at least 6, at least 7, at least 8, at least 10, at least 15, at least 20, or at least 30 nodes. In some embodiments, the second subset of control nodes comprises no more than 50, no more than 30, no more than 20, no more than no more than 10, no more than 8, no more than 5, no more than 4, or no more than 3 nodes. In some embodiments, the second subset of control nodes consists of from 2 to 5, from 2 to 10, from 4 to 15, from 10 to 25, from 12 to 40, or from 20 to 50 nodes. In some embodiments, the second subset of control nodes comprises falls within another range starting no lower than 2 nodes and ending no higher than 50 nodes.

In some embodiments, for a respective candidate drug therapy, the candidate off-target treatment model comprises a plurality of nodes that consists of the second subset of intervention nodes and the second subset of control nodes. In other words, in some embodiments, each node in the plurality of nodes for the candidate off-target treatment model, other than the second subset of intervention nodes, is a control node.

As described above, in some embodiments, a respective control node in the second subset of control nodes further comprises a corresponding control constant, in a set of control constants. In some embodiments, the set of control constants comprises at least 2, at least 3, at least 4, at least 5, at least 6, at least 7, at least 8, at least 10, at least 15, at least 20, at least 30, or at least 50 control constants. In some embodiments, the set of control constants comprises no more than 100, no more than 50, no more than 30, no more than 20, no more than no more than 10, no more than 8, no more than 5, no more than 4, or no more than 3 control constants. In some embodiments, the set of control constants consists of from 2 to 5, from 2 to 10, from 4 to 15, from 10 to 25, from 12 to 40, from 20 to 60, or from 50 to 100 control constants. In some embodiments, the set of control constants comprises falls within another range starting no lower than 2 control constants and ending no higher than 100 control constants.

In some embodiments, the set of control constants comprises, for each respective control node in the second subset of control nodes, a corresponding one or more control constants for the corresponding control function (e.g., each function is associated with at least one constant). In some embodiments, a control function comprises one or more control constants. In some embodiments, a control function comprises a plurality of control constants (e.g., a function can be associated with more than one constant).

In some embodiments, a control node, control function, and/or control constant thereof for a control node of a respective candidate off-target treatment model is selected from any of the embodiments disclosed elsewhere herein, including but not limited to any of the control nodes, control functions, and/or control constants disclosed with reference to any of the reference off-target treatment models (see, e.g., the section entitled “Obtaining reference off-target treatment models,” above), as will be apparent to one skilled in the art.

### Candidate Time-Course Progression and Candidate Signatures

In some embodiments, for each respective candidate drug therapy in the set (e.g., plurality) of candidate drug therapies, the candidate time-course progression comprises one or more candidate signatures.

In some embodiments, the candidate time-course progression comprises a plurality of candidate signatures. In some embodiments, the plurality of candidate signatures includes at least 2, at least 3, at least 4, at least 5, at least 6, at least 7, at least 8, at least 10, at least 15, at least 20, or at least 30 signatures. In some embodiments, the plurality of candidate signatures comprises no more than 50, no more than 30, no more than 20, no more than no more than 10, no more than 8, no more than 5, no more than 4, or no more than 3 signatures. In some embodiments, the plurality of candidate signatures consists of from 2 to 5, from 2 to 10, from 4 to 15, from 10 to 25, from 12 to 40, or from 20 to 50 signatures. In some embodiments, the plurality of candidate signatures comprises falls within another range starting no lower than 2 signatures and ending no higher than 50 signatures.

Referring to Block 2242, in some embodiments, the first candidate signature for the first biological intermediate in the first plurality of biological intermediates comprises, for each timepoint in a plurality of timepoints, a corresponding concentration of the first biological intermediate.

In some embodiments, a candidate signature is a measure of activity, amount, or change thereof, for a respective biological intermediate in the first plurality of biological intermediates for the respective off-target biological network. In some embodiments, any of the embodiments for reference time-course progressions and/or reference signatures thereof described herein are contemplated for use in generating a candidate time-course progression and/or candidate signature thereof, as will be apparent to one skilled in the art. In some embodiments, the candidate time-course progression comprises a plot of enzyme kinetics for at least one enzyme or biological intermediate thereof.

### Determination of Candidate Drug Therapies

Referring to Block 2248, in some embodiments, the method further includes determining, in the set (e.g., plurality) of candidate drug therapies 2152, one or more candidate drug therapies having a corresponding first candidate signature 2158 that matches the first reference signature 2142.

In some implementations, the method advantageously determines whether a candidate drug therapy has an effect on a biological intermediate of interest that is similar to that of the reference therapy. This effect can be a beneficial or an adverse effect. In some embodiments, the method further includes selecting the one or more candidate drug therapies that match the first reference for further development or synthesis. In some embodiments, the method further includes using one or more candidate signatures that match one or more reference signatures (e.g., for one or more biological intermediates) to select the one or more candidate drug therapies. In some embodiments, the method further includes using a plurality of candidate signatures that match one or more reference signatures (e.g., for a plurality of biological intermediates) to select the one or more candidate drug therapies. For instance, in some embodiments, it is desirable to evaluate the effect of a candidate drug therapy on multiple biological intermediates of interest, or on all of the intermediates in a particular biological pathway.

Referring to Block 2249, in some embodiments, the method further includes synthesizing, for each respective candidate drug therapy having a corresponding first candidate signature that matches the first reference signature, the respective candidate drug therapy.

Referring to Block 2250, in some embodiments, the method further includes using the one or more candidate drug therapies as treatment for a target biological network.

### Systems and Methods for Developing an Alternative Drug Therapy

Also described herein are systems and methods for developing an alternative drug therapy using characteristics of an existing drug therapy to produce a similar pathway behavior. The following description is presented to enable any person skilled in the art to make and use the invention as claimed and is provided in the context of the particular examples discussed below, variations of which will be readily apparent to those skilled in the art. In the interest of clarity, not all features of an actual implementation are described in this specification. It will be appreciated that in the development of any such actual implementation (as in any development project), design decisions must be made to achieve the designers' specific goals (e.g., compliance with system- and business-related constraints), and that these goals will vary from one implementation to another. It will also be appreciated that such development effort might be complex and time-consuming but would nevertheless be a routine undertaking for those of ordinary skill in the field of the appropriate art having the benefit of this disclosure. Accordingly, the claims appended hereto are not intended to be limited by the disclosed embodiments but are to be accorded their widest scope consistent with the principles and features disclosed herein.

In some embodiments, the reference drug therapy is an existing drug therapy for a targeted biological network, and the method further includes determining a candidate drug therapy by a process comprising obtaining an existing-treatment mathematical model for the targeted biological network comprising a plurality of nodes.

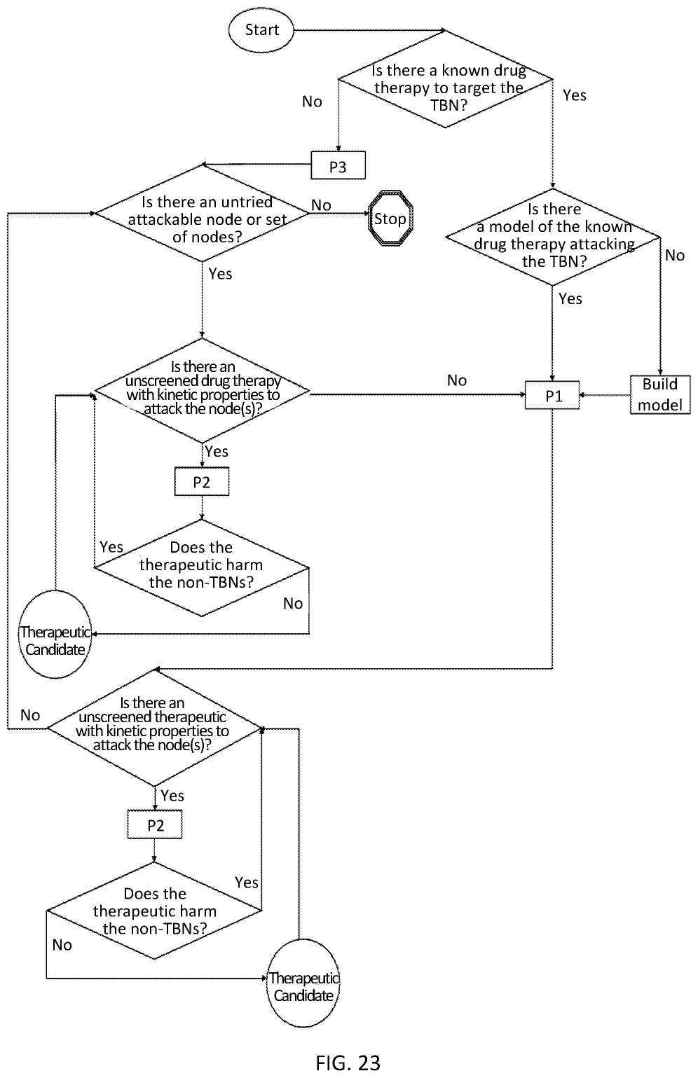

For instance, FIG. 23 illustrates an example schematic for developing a candidate drug therapy using characteristics of an existing drug therapy and/or determining an effect of a candidate drug therapy, in accordance with an embodiment of the present disclosure. P1: evaluation of reference drug therapy; P2: evaluation of candidate drug therapy; P3: de novo modeling of perturbation in targeted biological network; TBN: targeted biological network; non-TBN: non-targeted biological network (e.g., off-target biological network).

In some embodiments, the existing-treatment mathematical model comprises a first subset of intervened-upon nodes in the plurality of nodes, each respective intervened-upon node in the first subset of intervened-upon nodes comprising (i) a corresponding existing-treatment intervention function that represents one or more chemical interconversions, responsive to the existing drug therapy, at the respective intervened-upon node and (ii) a corresponding existing-treatment intervention constant, from a set of existing-treatment intervention constants, for the respective existing-treatment intervention function. In some embodiments, the existing-treatment mathematical model further includes a second subset of no-treatment nodes in the plurality of nodes, each respective no-treatment node in the second subset of no-treatment nodes comprising a corresponding no-treatment function that represents one or more chemical interconversions, responsive to an absence of the existing drug therapy, at the respective no-treatment node. In some embodiments, the existing-treatment mathematical model produces a corresponding existing-treatment time-course progression for the targeted biological network that comprises at least a first signature that is related to an existing-treatment outcome of the targeted biological network.

In some embodiments, the process further includes developing, for each respective permutation in a plurality of permutations, a respective new-treatment mathematical model of the targeted biological network. In some embodiments, the respective new treatment mathematical model comprises a third subset of intervened-upon nodes in the plurality of nodes, each respective intervened-upon node in the third subset of intervened-upon nodes comprising (i) a corresponding new-treatment intervention function that represents one or more chemical interconversions at the respective intervened-upon node and (ii) a corresponding new-treatment intervention constant, from a corresponding set of new-treatment intervention constants, for the respective new-treatment intervention function. In some embodiments, the respective new treatment mathematical model further includes a fourth subset of no-treatment nodes in the plurality of nodes, each respective no-treatment node in the fourth subset of no-treatment nodes comprising a corresponding no-treatment function that represents one or more chemical interconversions at the respective no-treatment node.

In some embodiments, the respective new-treatment mathematical model produces a corresponding new-treatment time-course progression for the targeted biological network comprising at least a corresponding second signature related to a respective new-treatment outcome of the targeted biological network. In some embodiments, the first subset of intervened-upon nodes comprises at least one node not in the third subset of intervened-upon nodes, and the third subset of intervened-upon nodes comprises at least one node not in the first subset of intervened-upon nodes.

In some embodiments, the process further includes synthesizing, for each respective permutation in the plurality of permutations having a corresponding second signature that matches the first signature for the existing-treatment time-course progression, a corresponding drug regimen for each respective new treatment intervention constant in the corresponding set of new-treatment intervention constants, thereby obtaining a set of drug regimens together forming a new drug therapy for the respective permutation, wherein at least one drug regimen in the set of drug regimens is not included in the existing drug therapy.

In some embodiments, the method further includes using the new drug therapy as a candidate drug therapy for the off-target biological network.

As described above, in some embodiments, an existing-treatment model is obtained by any suitable means, including but not limited to models that are previously generated, obtained from a database, and/or generated using experimental values for biological intermediates. In some embodiments, the existing treatment is selected from the group consisting of: inhibitors, chemicals, small molecules, agonists, polypeptides, proteins, RNA, DNA, and/or lipids.

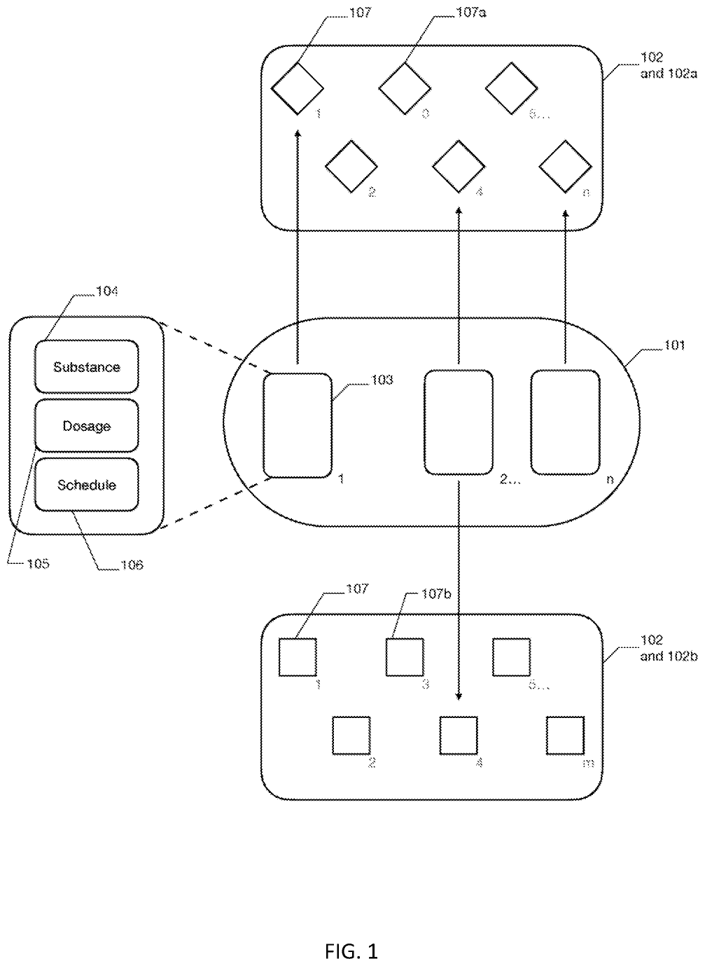

FIG. 1 illustrates a drug therapy 101 interacting with a biological network 102. Within the context of this disclosure, biological network 102 can be a target biological network (TBN) 102a or a non-targeted biological network (non-TBN) 102b. TBN 102a can include, but is not limited to, all or a portion of a pathogen or a disease. TBN 102a can be multicellular, single cellular, or even RNA or DNA. Example categories of TBN 102a can include parasitic animals, bacteria, viruses, or fungi. Specific examples can in include E-coli, COVID-19, or cancer. Non-TBN 102b, for purposes of this disclosure, is biological network 102 within a host or organism in a mutualistic relationship with the host.

This disclosure describes systems and method for developing one or more drug therapies 101 that disrupt TBN 101a. Drug therapy 101 is any one or more drug regimens 103, other than food, that is/are used to prevent, diagnose, treat, or relieve symptoms of a disease or abnormal condition. Further, for purposes of this disclosure, drug regimen 103 can be defined by substance 104. Substance 104, for purposes of this disclosure is a particular kind of matter with uniform properties. Additionally, drug regimen 103 can be defined by dosage 105, and schedule 106. Schedule 106, in one embodiment, can be defined by a period and/or duration (e.g., every 8 hours for 3 days). In some embodiments of drug regimen 103, dosage 105 can vary with schedule 106, such as increasing or decreasing over time. For purposes of this disclosure, dosage 105 can be described in absolute amounts, amounts meant to be scaled by other patient-specific information such as weight, age, maturity, etc., as intended concentrations, or as any other method to describe dosage known in the art.

Biological networks 102 comprise nodes 107. For purposes of this disclosure, nodes 107 are aspects of biological networks 102 upon which drug therapy 101 could potentially intervene-upon such as by accelerating, decelerating, preventing, or initiating a chemical interconversion, within biological network 102. Further, for purposes of this disclosure, nodes 107 of TBN 102a are target nodes 107a, and nodes 107 of non-TBN 102b are non-target nodes 107b.

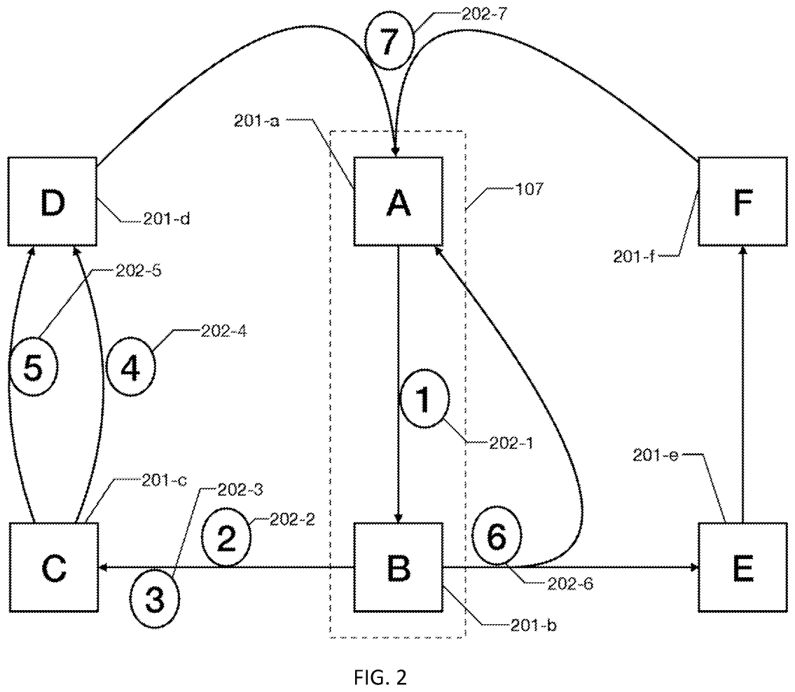

FIG. 2 illustrates an exemplary reaction model 200, in particular a pre-treatment reaction model 200a of biological network 102. For purposes of this disclosure, pre-treatment model 200a is a reaction model of the biological network 102 when biological model 102 is not undergoing treatment by drug therapy 101. As shown in FIG. 2, reaction model 200 represents a network of nodes 107, each a chemical interconversion of chemicals 201 in biological network 102. Such chemical interconversions are often facilitated by proteins 202.

Protein 202 can be and often is an enzyme. Furthermore, chemical 201 can be a non-enzymatic protein 202. Reaction model 200 as shown, represents biological network 102 comprising seven nodes 107, as follows:
Node 1: Chemical A converts to chemical B, facilitated by protein 1.
Node 2,3: Chemical B converts to chemical C, facilitated by protein 2 and protein 3.
Node 4: Chemical C converts to chemical D, facilitated by protein 4
Node 5: Chemical C converts to chemical D, facilitated by protein 5
Node 6: Chemical B converts to chemical A and chemical E by protein 6.
Node 7: Chemical D and F together convert to chemical A, facilitated by protein 7.
Node X_{E}: Chemical E converts to chemical F without a modeled protein.

A person of ordinary skill in the art will recognize that not all chemical interconversions occurring within biological network 102 need be represented in reaction model 200. Furthermore, within each chemical interconversion represented within reaction model 200, not all chemicals 201 or proteins 202 involved in the chemical interconversion need be modeled within reaction model 200. For example, in process 1, A+Z₁→B may in fact be A+x₁+Z₁→B+y₁ wherein x₁ is a set of non-modeled reactants for processes 1, and y₁ is a set of non-modeled products for processes 1.

FIG. 3 illustrates a time-course progression 300 of biological network 102, specifically an actual time-course progression 300z. For purpose of this disclosure, actual time-course progression 300z is a time-course progression of concentrations of chemicals 201 experimentally-measured.

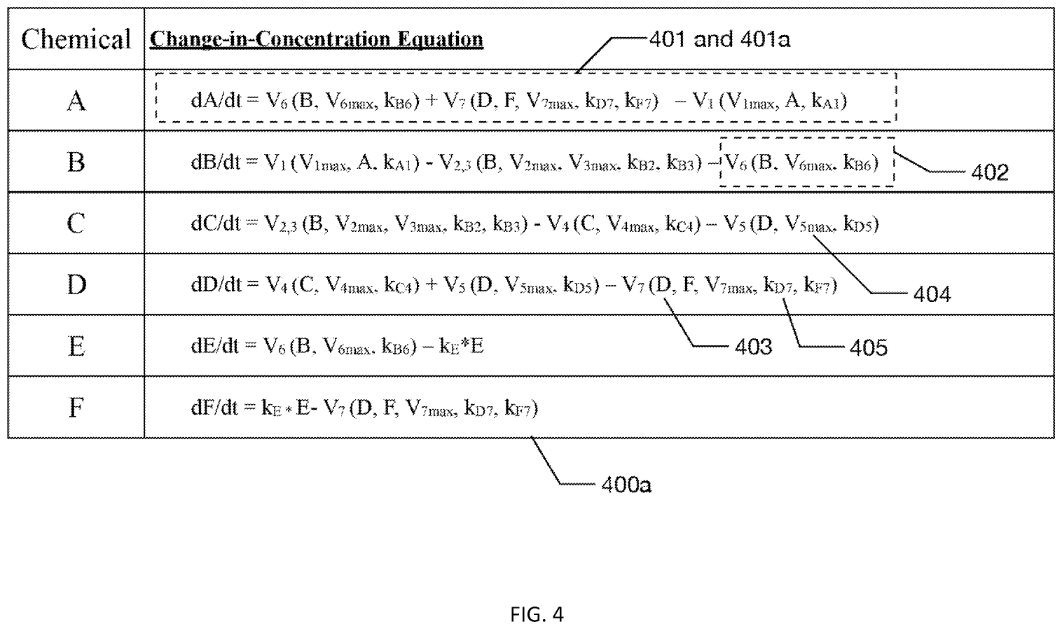

FIG. 4 illustrates a mathematical model 400 of biological network 102, in particular a pre-treatment mathematical model 400a. For purposes of this disclosure, mathematical model 400 relates to reaction model 200 and comprises equations 401 that together describe the behavior of biological network 102 with sufficient accuracy so as to be able to accurately predict the behavior of biological network 102 to which reaction model 200 relates. Further, for purposes of this disclosure, pre-treatment mathematical model 400a relates to pre-treatment reaction model 200a and comprises equations 401 that together describe the behavior of biological network 102 when not undergoing treatment by any drug therapy 101. Equations 401, in one embodiment, can comprise change-in-concentration equations 401a that each describe the rate of change in concentrations of a particular chemical 201 within reaction model 200. For example, change-in-concentration equations 301a relating to chemical A is as follows: dA/dt=V6(B, V6max, kB6)+V7(D, F, V7max, kD7, kF7)−V1(V1max, A, kA1).

Change-in-concentration equations 401a can comprise node-velocity functions Vₙ ( ) 402 that describe the behavior of nodes 107 and are denoted in FIG. 4 as functions of various variables. For example, variables A through F each represent a concentration 403 of a corresponding chemicals 201 A-F in reaction model 201. V_{n max} represents a maximum node velocity 404 at which protein 202 can convert reactant(s) to product(s). Node velocity functions 402 each can be used to determine node velocity, and node velocities can be used to calculate changes in concentration of chemicals 201. One of ordinary skill in the art will recognize that node velocities can be modeled with a Michaelis-Menten equation. As an example, node velocity V₁ can be modeled with the equation V₁=(V₁ₘₐₓ*A)/(k_{A1}+A). In such equation, k_{A1} is a rate constant 405 specific to chemical A and protein 1. One of ordinary skill in the art will recognize that Both V₁ₘₐₓ and k_{A1} can be determined experimentally. These values can also be estimated. Furthermore, proteins 202 can operate on multiple chemicals or output multiple chemicals, leading to more complicated equations. Similarly, some nodes may require multiple proteins which can also lead to more complicated equations.

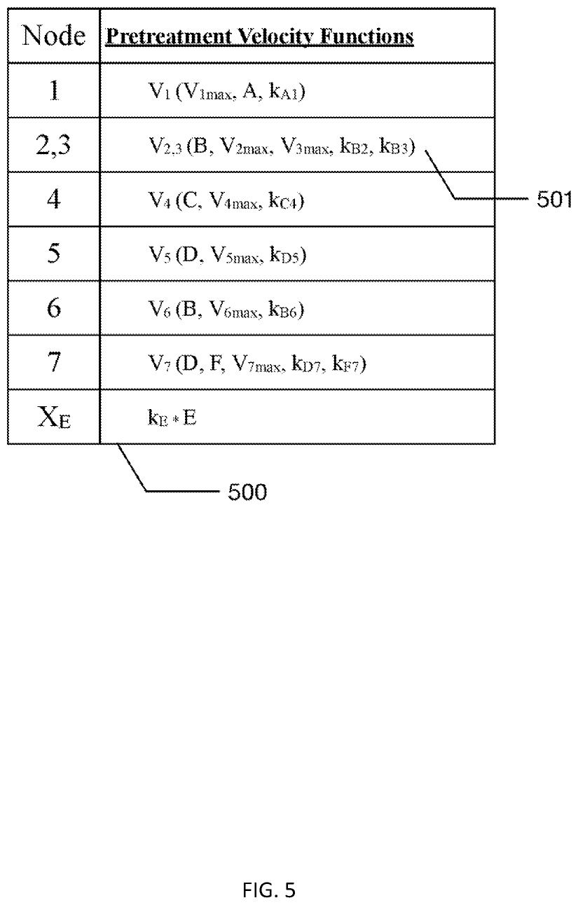

FIG. 5 illustrates a set of pretreatment node-velocity functions 500 for a pre-treatment mathematical model 400a model of biological network 102 not undergoing treatment, each node-velocity function is a pre-treatment velocity functions 501. For purposes of this disclosure, pre-treatment velocity function 501 is a node velocity function that models node 107 when such node is not being intervened-upon by drug regimen 103.

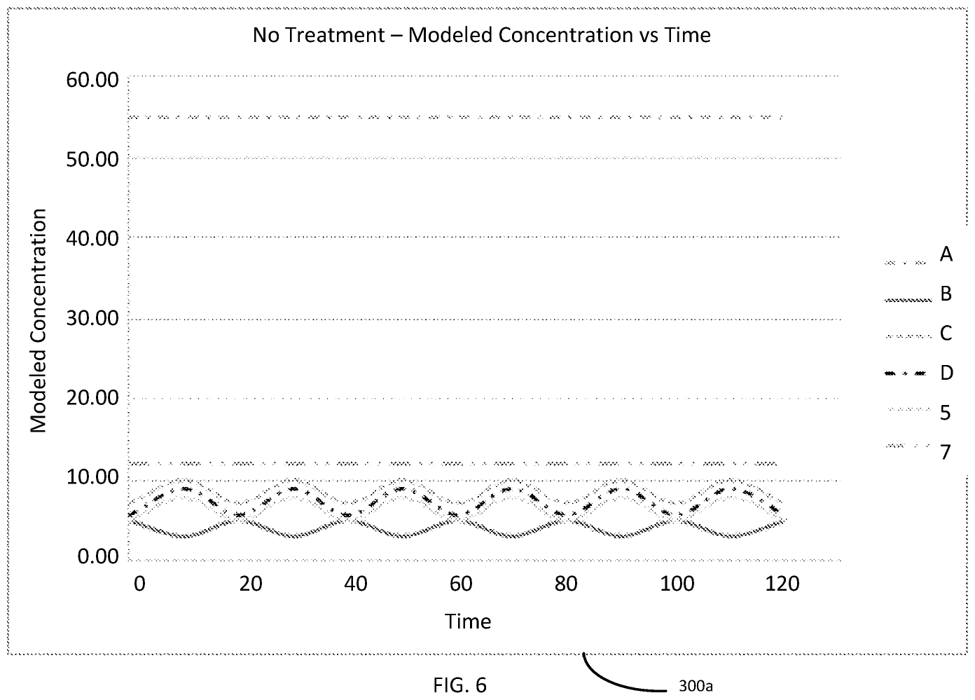

FIG. 6 illustrates a pre-treatment time-course progression 300a. One purpose of pre-treatment mathematical model 300a is to generate pre-treatment time-course progression 300a. For purpose of this disclosure, pre-treatment time course progression is a time course progression of pre-treatment mathematical model 300a and is intended to model with sufficient accuracy actual time-course progression 300z.

Time-course progressions 300 comprise levels of chemical concentrations of chemicals 201 within measured or modeled biological network 102 as a function of time. Pre-treatment time-course progression 300a models levels of chemical concentrations of chemicals 201 within biological network 102 as a function of time, prior to any treatment by drug therapy 101. Absent treatments, concentrations of chemicals 201 may vary in cycles, but otherwise typically remain within predictable, constrained levels for sustained periods within a life cycle of biological network 102. That is not to say that concentrations will remain constrained for an entire life cycle, but that during periods, concentrations will remain constrained, and that over changes from one period to the next remain substantially predictable. An important function of pretreatment time-course progression 400a is that it can establish baseline dynamics of biological network 102.

FIG. 7 illustrates an existing-treatment reaction model 200b. For purposes of this disclosure, existing treatment reaction model 200b is a reaction model that models how an existing drug therapy interacts with biological network 102. Existing-treatment reaction model 200b comprises at least one existing-treatment intervened-upon node 107a. Existing-treatment intervened-upon node 107a is intervened upon by drug regimen 103. Remaining nodes 107 that are not intervened upon are modeled as an untreated node 107b with a pre-treatment velocity function 501. It should be noted that existing drug therapy 101 is not exclusive only to drug therapies 101 that have reached the market but instead include any drug therapies previously considered for use on biological network 102 and lead to an outcome. In the case of target biological network 102a, examples of an outcome include but are not limited to causing target biological network 102a to die, rendering target biological network 102a unable to reproduce, or substantially undermine target biological network 102a such that other conditions or forces such as an immune system can kill target biological network 102a. In the case of a non-target biological network 102b, examples of an outcome can include strengthening non-target biological network 102b or rending non-target biological network 102b resistant or immune to some condition.

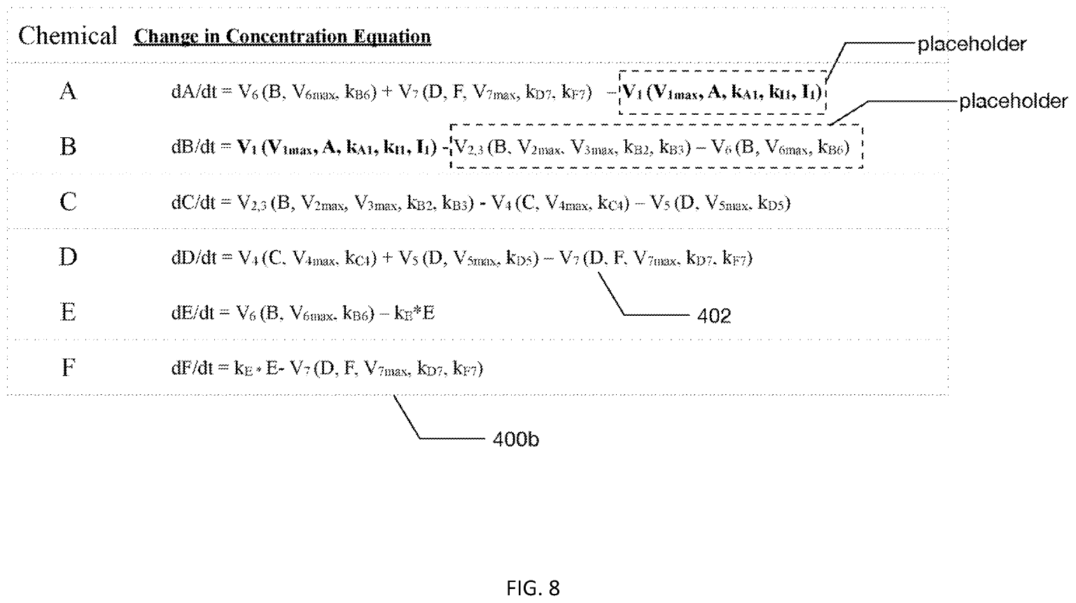

FIG. 8 illustrates an existing-treatment mathematical model 400b. In existing-treatment mathematical model 400b, each node-velocity function 402 modeling an existing-treatment intervened-upon node 107a can be an intervention function 402a. Types of intervention functions fall into two primary categories: an inhibition function or an acceleration function. Inhibition functions model the slowing or substantially stopping of a chemical interconversion at existing-treatment intervened-upon node 107a. Conversely, an acceleration function model the starting or speeding up of a chemical interconversion at existing treatment intervened-upon node 107a.

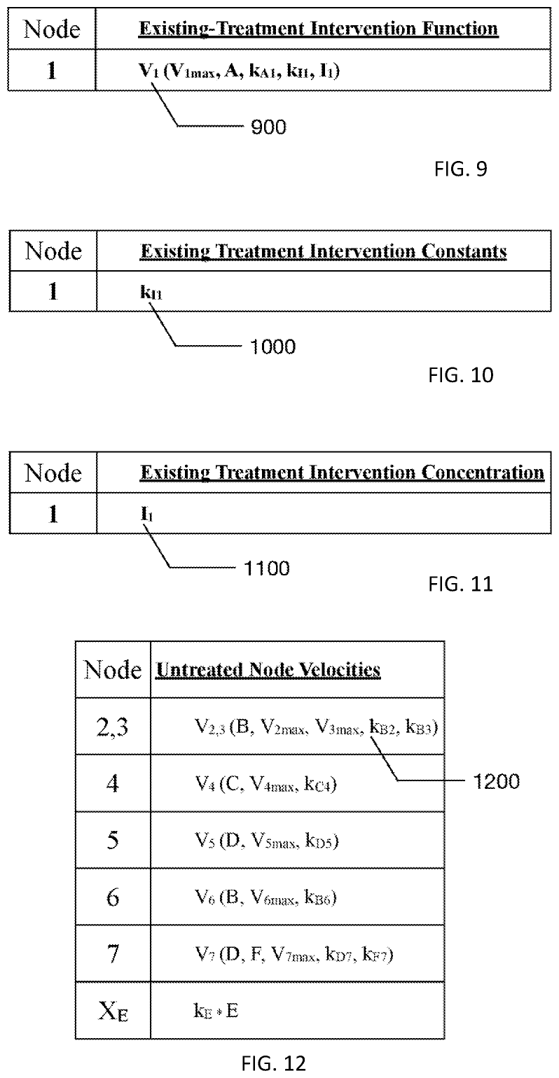

FIG. 9 illustrates a first set of existing-treatment intervention functions 900. As shown in FIG. 9, the first set 900 can be a set of one or more existing-treatment intervention functions.

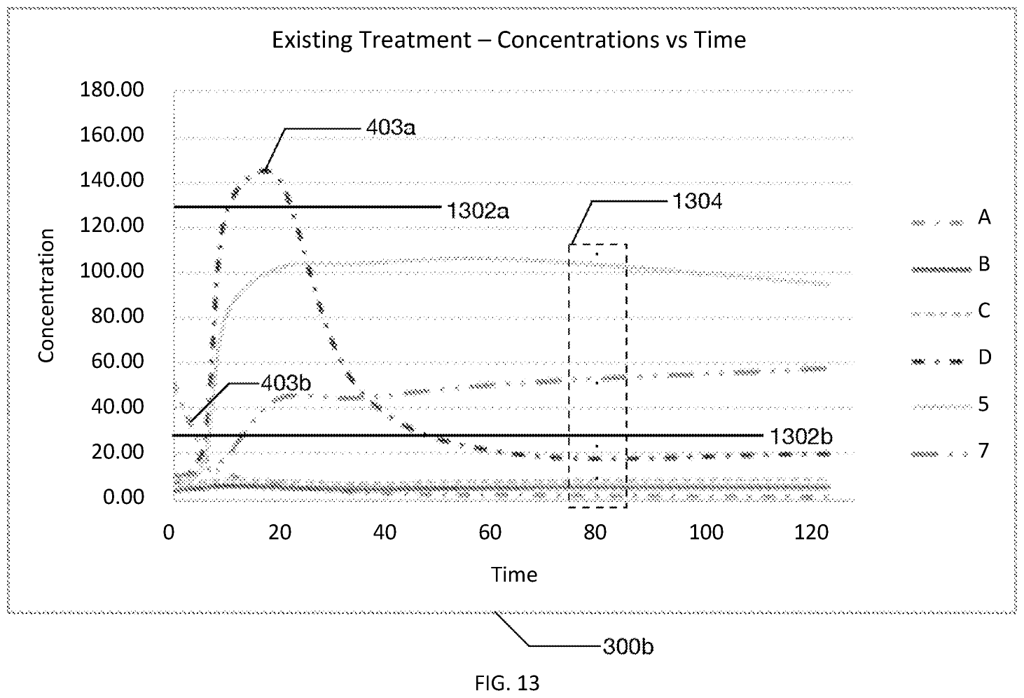

FIG. 10 illustrates a set of existing-treatment intervention constants 1000. Each intervention function 402a can have one or more intervention constants 1001 related to one drug regimen 103 of existing drug therapy 101 related to existing-treatment mathematical model 400b. An intervention constant is 1001 a number that represents the effect on the speed of a node that related drug regimen 103 has on node 107.

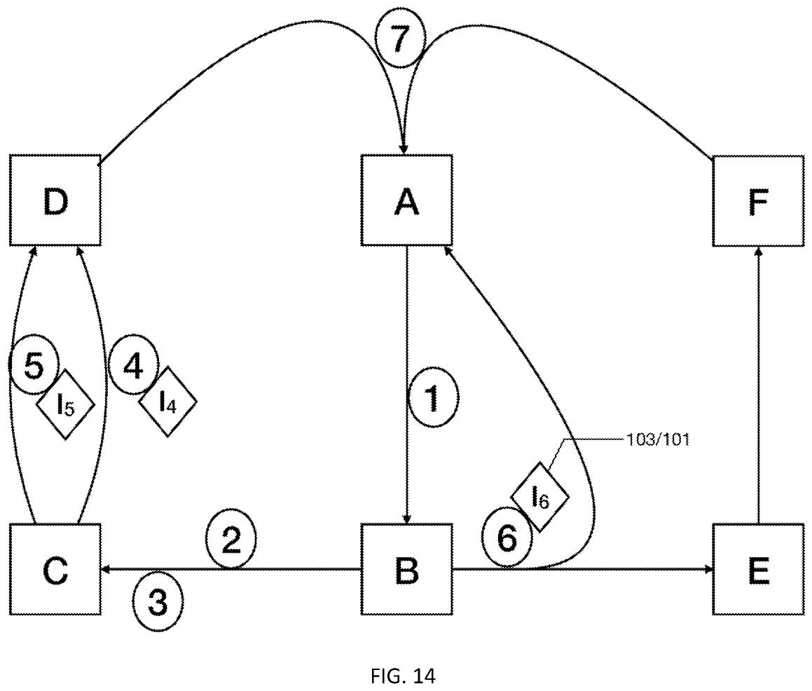

FIG. 11 illustrates a set of existing-treatment intervention concentrations 1100. Each intervention function 402a can comprise a concentration constant 1102 that models a concentration of one drug regimen 103 of existing drug therapy 101. The set of existing-treatment intervention concentrations 1100 comprises these concentration constants 1102.

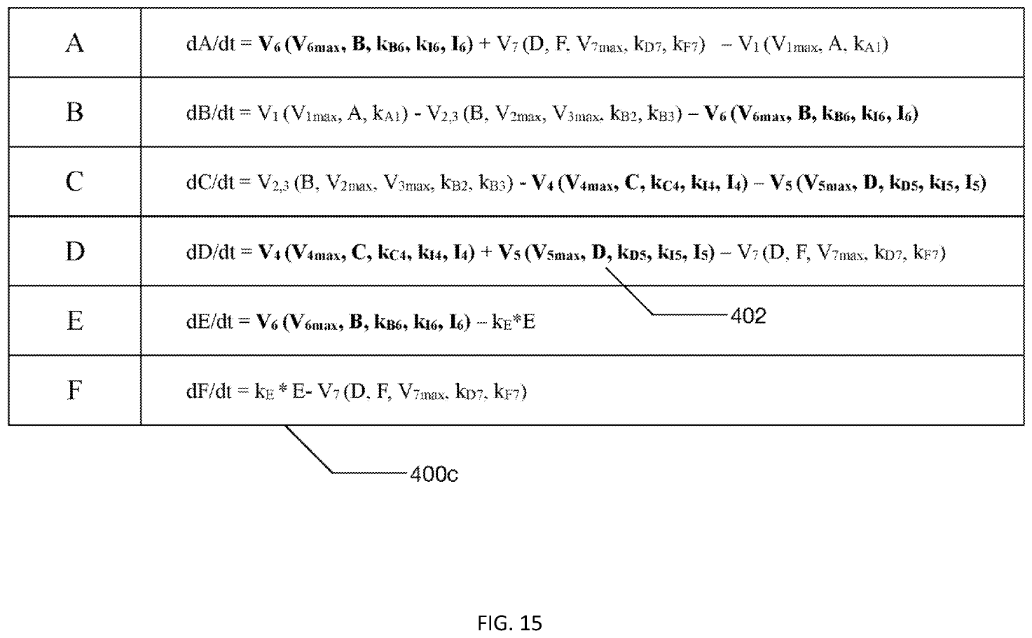

FIG. 12 illustrates a first set of untreated node velocity functions 1200. Within existing-treatment mathematical model 400b, untreated nodes 107b are modeled with pretreatment velocity functions 501. The first set of untreated node velocity functions 1200 comprises each of these pretreatment velocity functions 501.

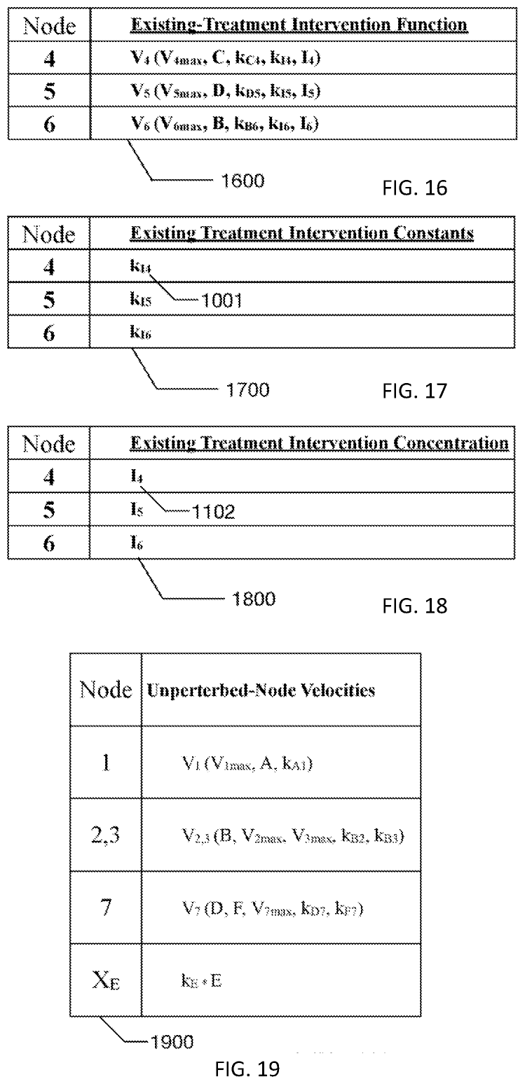

FIG. 13 illustrates an existing-treatment time-course progression 300b. Existing time-course progression 300b can comprise a time-course progression signature 1301 that predict outcomes as described above. Time-course progression signature 1301 can comprise one or more attributes. In one embodiment, time-course progression signature 1301 can be a first chemical concentration 403a of a first chemical 201 reaches a threshold 1302. In another embodiment, time-course progression signature 1301 can comprises a sequence of events. For example, the sequence of events can be defined at least in part by a first chemical 201a having a first chemical concentration 403a that meets or passes a first threshold 1302a followed by a second chemical 201b having a second chemical concentration 403b that meets or passes a second threshold 1302b. As another example, the sequence of events the defined at least in part by first chemical 201a having first chemical concentration 403a that meets or passes first threshold followed by the first chemical concentration a that meets or passes second threshold 1302b. In another embodiment, the time-course progression signature 1301 can comprise first chemical concentration 403a of first chemical 201a meeting or passing first threshold while second chemical concentration 403b of second chemical 201 is meeting or exceeding second threshold 1302b. In another embodiment, the time-course progression signature can comprise chemical concentrations 403 of chemicals 201 of a set of chemicals 1303 entering a range such that the chemical concentrations together do not deviate from a set of target concentrations 1304 for each of chemical 201 by equal to or more than a deviation threshold. In such embodiment, the deviation between the set of chemical concentrations 1303 and the set of target concentrations 1304 can be determined using a root-mean squared calculation.

In one embodiment, a set of parameters can be found for a new drug therapy such that the new drug therapy produces an outcome similar to an existing drug therapy. In a first step, the method can comprise replacing, within a mathematical model any intervention functions related to an existing drug therapy with untreated-node functions related to the mathematical model, if the mathematical model has any such intervention functions. In a next step, the method can comprise choosing parameter ranges for each of a plurality of parameters.

In some embodiments, the plurality of parameters comprises at least 2, at least 3, at least 4, at least 5, at least 6, at least 7, at least 8, at least 10, at least 15, at least 20, or at least 30 parameters. In some embodiments, the plurality of parameters comprises no more than 50, no more than 30, no more than 20, no more than no more than 10, no more than 8, no more than 5, no more than 4, or no more than 3 parameters. In some embodiments, the plurality of parameters consists of from 2 to 5, from 2 to 10, from 4 to 15, from 10 to 25, from 12 to 40, or from 20 to 50 parameters. In some embodiments, the plurality of parameters comprises falls within another range starting no lower than 2 parameters and ending no higher than 50 parameters.

Next, the method can comprise, producing a time course progression for a new-treatment mathematical model for each permutation of a plurality of permutations, each time, using the permutation to produce the time course progression. Next, the method can comprise determining for each permutation whether its time-course progression comprises a time-course progression signature present in an existing-treatment time-course progression related to the existing drug therapy, the time-course progression signature related to an outcome of the existing drug therapy. Next, the method can comprise, for at least one permutation comprising the time-course progression signature, synthesizing substances having kinetic properties substantially matching kinetic parameters of that one permutation, to produce a new drug therapy.

In some embodiments, the plurality of permutations comprises at least 2, at least 3, at least 4, at least 5, at least 10, at least 15, at least 20, at least 30, at least 50, at least 100, at least 200, at least 300, or at least 500 permutations. In some embodiments, the plurality of permutations comprises no more than 1000, no more than 500, no more than 300, no more than 100, no more than 50, no more than 30, no more than 20, no more than no more than 10, no more than 8, no more than 5, no more than 4, or no more than 3 permutations. In some embodiments, the plurality of permutations consists of from 2 to 5, from 2 to 10, from 8 to 30, from 20 to 80, from 50 to 200, from 100 to 500, or from 500 to 1000 permutations. In some embodiments, the plurality of permutations comprises falls within another range starting no lower than 2 permutations and ending no higher than 1000 permutations.

In some embodiments, each permutation in the plurality of permutations comprises adjusting one or more parameters and obtaining a corresponding time-course progression and/or time-course progression signature, as described above.

In some embodiments, a parameter in the plurality of parameters is a number of nodes to be intervened upon, a designation of a set of nodes that can be intervened upon, a method of intervention, such as by speeding up or slowing down a chemical intervention within a node, one or more intervention equations for use in a treatment model, a set of range of one or more intervention constants for one or more intervention equations, one or more intervention concentrations, and/or spatial considerations.

In some embodiments, a parameter range comprises a designation of a set of nodes that can be intervened upon. In some embodiments, a parameter range comprises ways to intervene, such as by speeding up or slowing down a chemical intervention within a node. In some embodiments, a parameter range comprises a plurality of intervention equations to be considered. In some embodiments, a parameter range comprises a range for one or more intervention constants for one or more intervention equations. In some embodiments, a parameter range comprises ranges for acceptable intervention concentrations. In some a parameter, parameter range comprises spatial considerations.

Suitable parameters and/or parameters ranges contemplated for use in the present disclosure include, but are not limited to, any of the numbers, selection, types, or identity of nodes, methods of intervention, biological transitions, biological intermediates, intervention functions, intervention constants, control functions, control constants, timepoints, and/or concentrations, as described above, and any combinations or modifications thereof, as will be apparent to one skilled in the art.

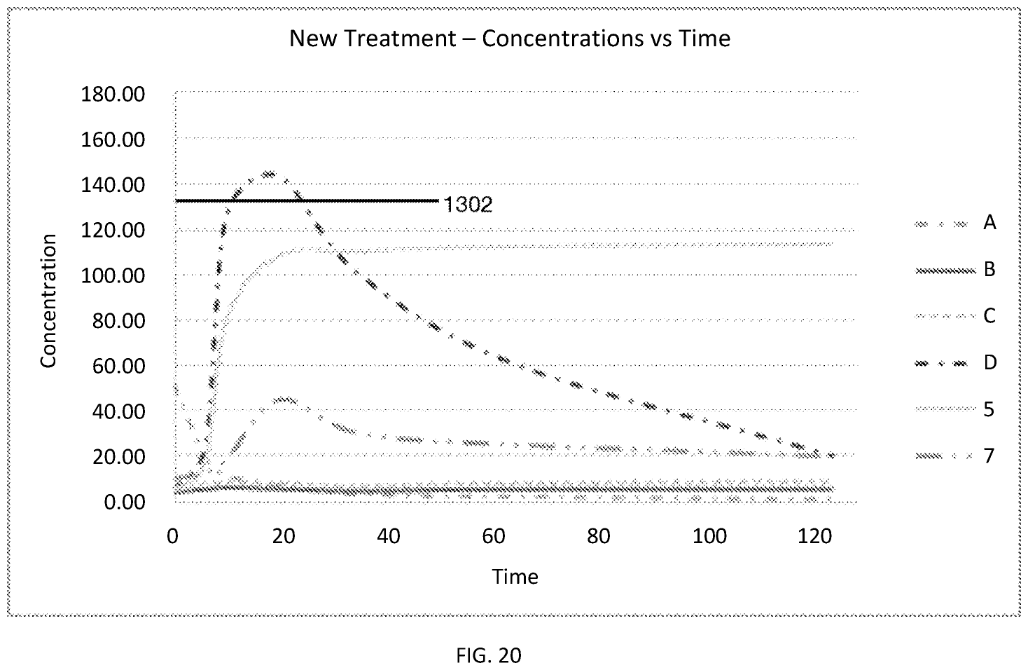

FIG. 14 illustrates a new-treatment reaction model 200c. For purposes of this disclosure, new-treatment reaction model 200c is a reaction model that models how a new drug therapy might interact with biological network 102. New-treatment reaction model 200c comprises at least one new-treatment intervened-upon node 107a. New-treatment intervened-upon node 107a is intervened upon by drug regimen 103 of a new drug therapy 101. Remaining nodes 107 that are not intervened upon are modeled as an untreated node 107b with a pre-treatment velocity function 501.

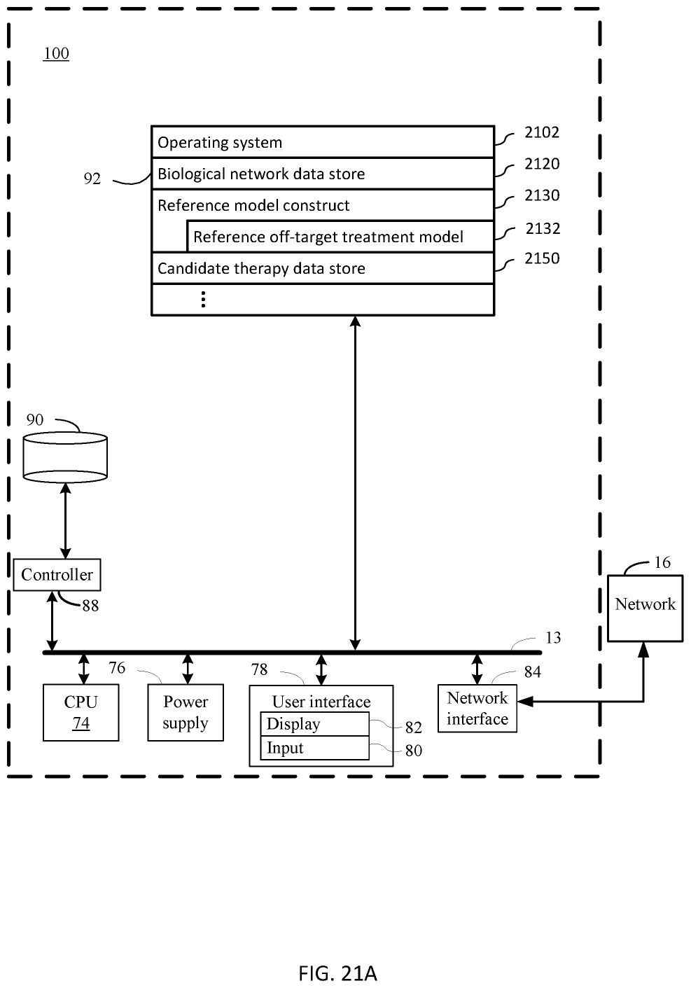

FIG. 15 illustrates new-treatment mathematical model 400c. In new-treatment mathematical model 400c, each node-velocity function 402 modeling new treatment intervened-upon node 107a can be an intervention function 402a. Types of intervention functions fall into two primary categories: an inhibition function or an acceleration function. Inhibition functions model the slowing or substantially stopping of a chemical interconversion at new-treatment intervened-upon node 107a. Conversely, an acceleration function model the starting or speeding up of a chemical interconversion at new-treatment intervened-upon node 107a.

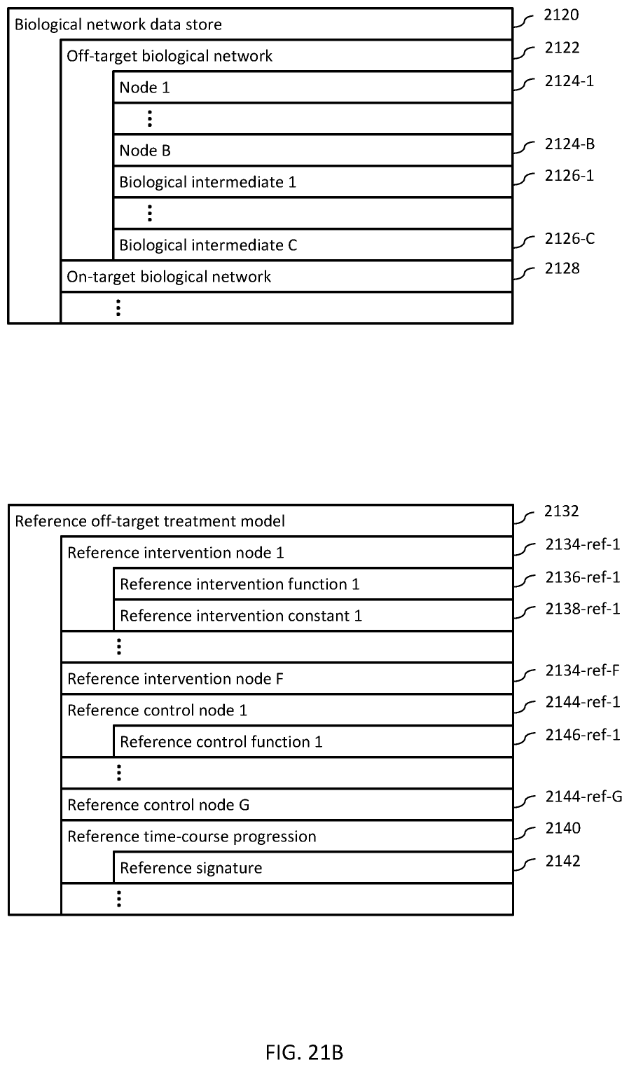

FIG. 16 illustrates a second set of new-treatment intervention functions 1600. As shown in FIG. 16, the second set 1600 can be a set of one or more new-treatment intervention functions.

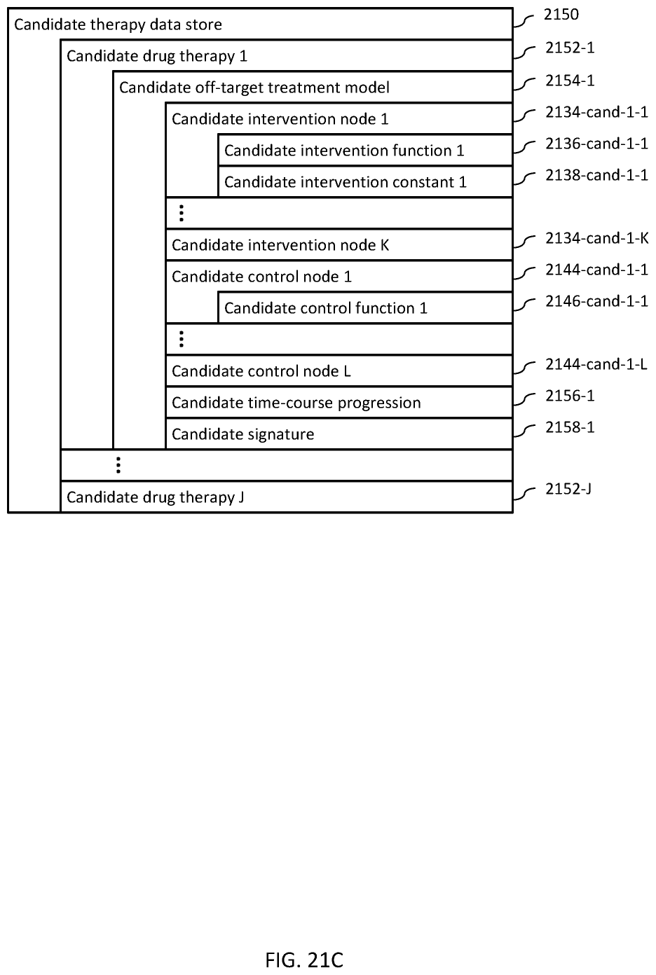

FIG. 17 illustrates a set of new-treatment intervention constants 1700. Each intervention function 402a can have one or more intervention constants 1001 related to one drug regimen 103 of new drug therapy 101 related to new-treatment mathematical model 400c. Intervention constant is 1001 a number that represents the effect on the speed of a node that related drug regimen 103 has on node 107.

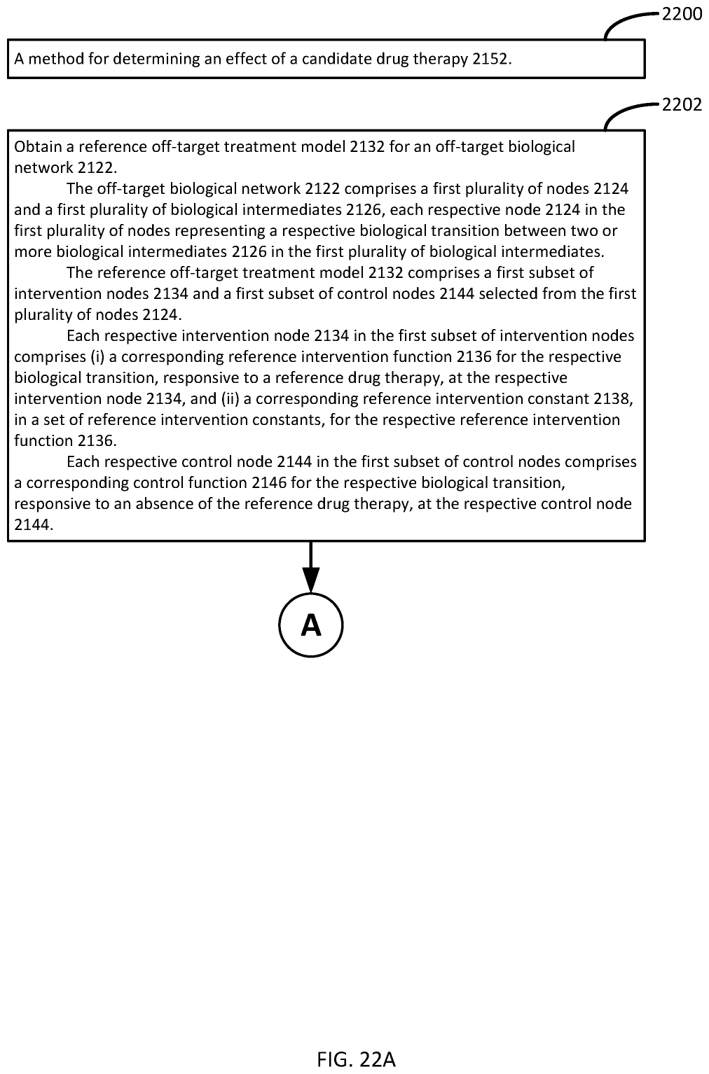

FIG. 18 illustrates a set of new-treatment intervention concentrations 1800. Each intervention function 402a can comprise concentration constant 1102 that models a concentration of one drug regimen 103 of new drug therapy 101. The set of existing-treatment intervention concentrations 1800 comprises these concentration constants 1102.

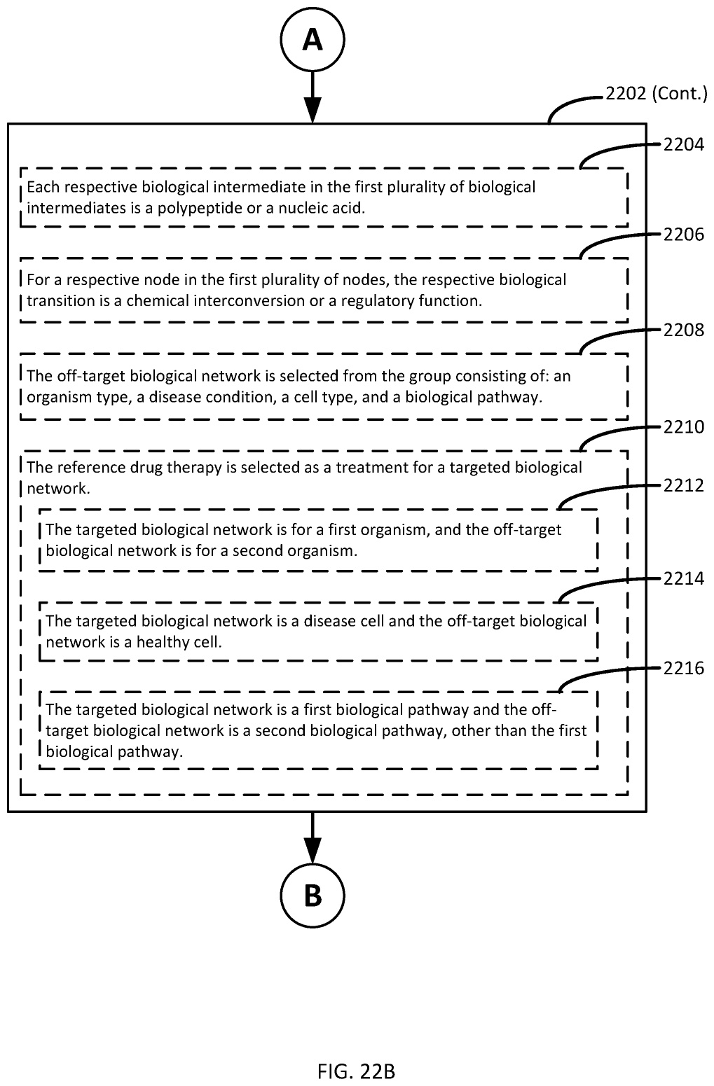

FIG. 19 illustrates a second set of untreated node velocity functions 1900. Within new-treatment mathematical model 400b, untreated nodes 107b are modeled with pretreatment velocity functions 501. The first set of untreated node velocity functions 1200 comprises each of these pretreatment velocity functions 501.

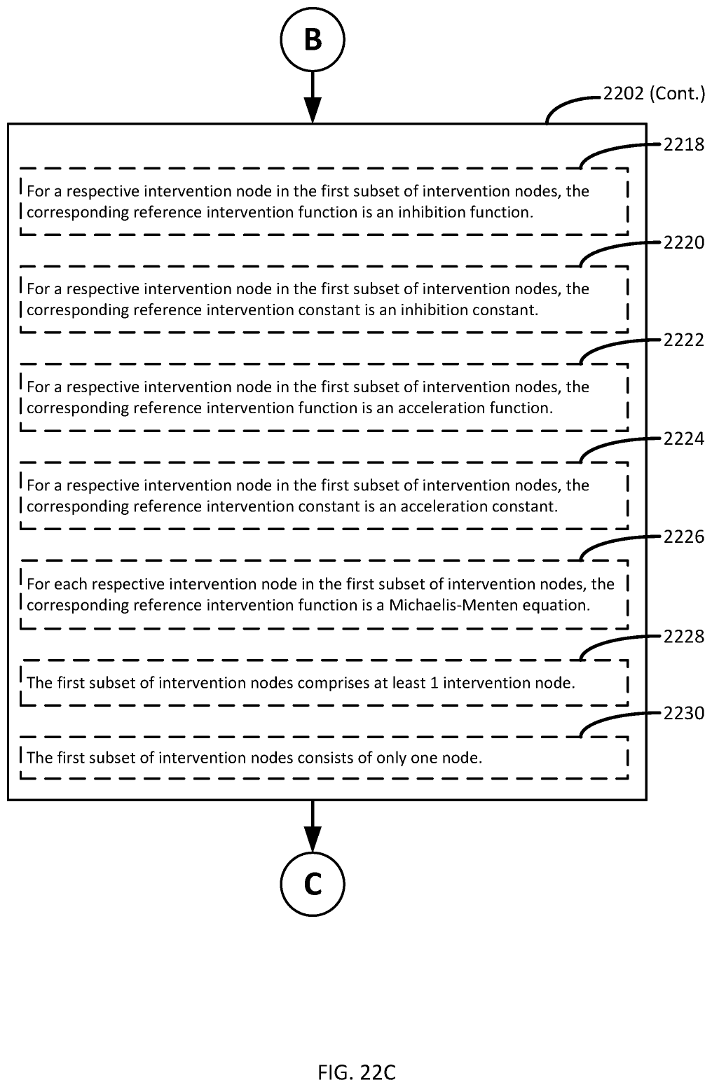

FIG. 20 illustrates a new-treatment time-course progression 300c. New time-course progression 300c can comprise time-course progression signature 1301 that predicts and is common, related to, or otherwise present in existing-treatment time-course progression 300b.

Accordingly, in some embodiments, for each respective intervened-upon node in the first subset of intervened-upon nodes, the corresponding existing-treatment intervention function is an inhibition function and the corresponding existing-treatment intervention constant is an inhibition constant. In some embodiments, for each respective intervened-upon node in the third subset of intervened-upon nodes, the corresponding new-treatment intervention function is an inhibition function (e.g., inhibition equation) and the corresponding new-treatment intervention constant associated with said inhibition function (e.g., equation) is an inhibition constant.

In some embodiments, for each respective intervened-upon node in the first subset of intervened-upon nodes, the corresponding existing-treatment intervention function is an acceleration function and the corresponding existing-treatment intervention constant is an acceleration constant. In some embodiments, for each respective intervened-upon node in the third subset of intervened-upon nodes, the corresponding new-treatment intervention function is an acceleration function (e.g., an acceleration equation) and the corresponding new-treatment intervention constant associated with said acceleration function (e.g., equation) is an acceleration constant.

In some embodiments, a corresponding no-treatment function for a respective no-treatment node is a Michaelis-Menten equation. In some embodiments, the first subset of intervened-upon nodes consists of only one node.

In some embodiments, the third subset of intervened-upon nodes (e.g., new treatment nodes) comprises a plurality of intervened-upon nodes. In some embodiments, there exist no nodes in common between the first subset of intervened-upon nodes (e.g., existing-treatment nodes) and the third subset of intervened-upon nodes (e.g., new treatment nodes).

In some embodiments, for each respective node in common between the first subset of intervened-upon nodes and the third subset of intervened-upon nodes, for each respective permutation in the plurality of permutations: (i) the corresponding existing-treatment intervention constant for the respective node in the existing-treatment mathematical model is different from (ii) the corresponding new-treatment intervention constant for the respective node in the respective new-treatment mathematical model corresponding to the respective permutation.

In some embodiments, the process further includes, prior to producing the new-treatment time-course progression, determining, for each respective parameter in a plurality of parameters, a corresponding parameter range for the respective parameter, the plurality of parameters comprising at least a first parameter that indicates one or more intervened-upon nodes in the plurality of nodes.

In some embodiments, each respective permutation in the plurality of permutations comprises a corresponding set of parameters in the plurality of parameters, and the method further comprises replacing, for each respective permutation in the plurality of permutations: (i) one or more intervened-upon nodes in the first subset of intervened-upon nodes with a corresponding no-treatment node in the fourth subset of no-treatment nodes, and/or (ii) one or more no-treatment nodes in the second subset of no-treatment nodes with a corresponding intervened-upon node in the third subset of intervened-upon nodes, based at least upon the corresponding parameter range for the first parameter in the plurality of parameters.

In some embodiments, the corresponding parameter range for a respective parameter in the plurality of parameters is selected from the group consisting of: a designation of one or more intervened-upon nodes, an acceleration of the chemical interconversion, an inhibition of the chemical interconversion, a plurality of intervention functions, a range of intervention constants, a range of abundance values for one or more biological elements in the targeted biological pathway, and a range of spatial considerations.

In some embodiments, the corresponding existing-treatment time-course progression for the existing-treatment mathematical model comprises, for each respective timepoint in a corresponding plurality of timepoints, for each respective biological element in a set of biological elements for the targeted biological network, a respective abundance value for the respective biological element. In some embodiments, for each respective permutation in the plurality of permutations, the corresponding new-treatment time-course progression for the respective new-treatment mathematical model comprises, for each respective timepoint in the corresponding plurality of timepoints, for each respective biological element in the set of biological elements for the targeted biological network, a respective abundance value for the respective biological element.

In some embodiments, each respective biological element in the set of biological elements is a chemical, a protein, RNA, or DNA, and the respective abundance value is a concentration.

In some embodiments, the first signature comprises at least a first abundance value for a first biological element in the set of biological elements that satisfies a first abundance threshold.

In some embodiments, the first signature further comprises a second abundance value for the first biological element that satisfies a second abundance threshold, and/or a third abundance value for a second biological element in the set of biological elements that satisfies a third abundance threshold.

In some embodiments, the first signature comprises a sequence of events (e.g., a plurality of timepoints).

In some embodiments, the first signature comprises, for each respective biological element in the set of biological elements, a corresponding range of abundance values that satisfies a corresponding deviation threshold for the respective biological element. In some embodiments, the corresponding deviation threshold for each respective biological element in the set of biological elements is determined using a root-mean squared calculation.

In some embodiments, the targeted biological network is all or a portion of a pathogen or a disease.

In some embodiments, the existing-treatment mathematical model consists of the first subset of intervened-upon nodes and the second subset of no-treatment nodes.

In some embodiments, for each respective permutation in the plurality of permutations, the new-treatment mathematical model consists of the third subset of intervened-upon nodes and the fourth subset of no-treatment nodes.

In some embodiments, the set of drug regimens comprises at least 1, 2, 3, 4, 5, or 6 drug regimens together forming the new drug therapy, wherein each respective drug regimen in the set of drug regimens is a respective drug defined by a corresponding dosage or a corresponding schedule.

In some embodiments, the set of drug regimens comprises at least 2, at least 3, at least 4, at least 5, at least 6, at least 7, at least 8, at least 10, at least 15, at least 20, or at least 30 drug regimens. In some embodiments, the set of drug regimens comprises no more than 50, no more than 30, no more than 20, no more than no more than 10, no more than 8, no more than 5, no more than 4, or no more than 3 drug regimens. In some embodiments, the set of drug regimens consists of from 2 to 5, from 2 to 10, from 4 to 15, from 10 to 25, from 12 to 40, or from 20 to 50 drug regimens. In some embodiments, the set of drug regimens comprises falls within another range starting no lower than 2 drug regimens and ending no higher than 50 drug regimens.

Yet another aspect of the present disclosure provides a method for developing a new drug therapy using characteristics of an existing drug therapy, the method comprising: (A) obtaining an existing-treatment mathematical model for a targeted biological network comprising a plurality of nodes. In some embodiments, the existing-treatment mathematical model comprises: an existing-treatment intervention equation that (i) models each respective existing-treatment intervened-upon node in a first subset of existing-treatment intervened-upon nodes in the plurality of nodes and (ii) comprises, for each respective existing-treatment intervened-upon node in the first subset of existing-treatment intervened-upon nodes, a respective existing-treatment intervention constant from a set of existing-treatment intervention constants. In some embodiments, the existing-treatment mathematical model further includes a no-treatment velocity equation that models each respective no-treatment node in a second subset of no-treatment nodes, where the second subset of no-treatment nodes comprises each respective node in the plurality of nodes not included in the first subset of existing-treatment intervened upon nodes. In some embodiments, the existing-treatment mathematical model generates an existing-treatment time-course progression comprising a corresponding time-course progression signature that is related to an existing-treatment outcome of the targeted biological network.

In some embodiments, the method further includes (B) developing a new-treatment mathematical model for the targeted biological network. In some embodiments, the new-treatment mathematical model comprises: a new-treatment intervention equation that (i) models each respective new-treatment intervened-upon node in a third subset of new-treatment intervened-upon nodes in the plurality of nodes and (ii) comprises one or more new-treatment intervention constants from a set of new-treatment intervention constants. In some embodiments, the new-treatment mathematical model further includes a no-treatment velocity equation that models each respective no-treatment node in a fourth subset of no-treatment nodes, where the fourth subset of no-treatment nodes comprises each respective node in the plurality of nodes not included in the third subset of new-treatment intervened upon nodes. In some embodiments, the new-treatment mathematical model generates a new-treatment time-course progression comprising the corresponding time-course progression signature found in the existing-treatment time-course progression of the existing treatment mathematical model.

In some embodiments, the third subset of new-treatment intervened-upon nodes comprises at least one node not in the first subset of existing-treatment intervened-upon nodes, and the first subset of existing-treatment intervened-upon nodes comprises at least one node not in the third subset of new-treatment intervened-upon nodes.

In some embodiments, the method further includes (C) synthesizing, for each respective new-treatment intervention constant in the one or more new-treatment intervention constants, a corresponding drug regimen, thereby producing a new drug therapy comprising one or more drug regimens.

In some embodiments, the method further includes treating a disease or abnormal condition using the new drug therapy.

Still another aspect of the present disclosure provides a method for developing a new drug therapy using characteristics of an existing drug therapy, the method comprising the following steps: developing a new-treatment mathematical model of a targeted biological network and synthesizing a drug therapy based on the new-treatment mathematical model.

The new treatment mathematical model can be capable of producing a new-treatment time-course progression comprising a time-course progression signature found in an existing-treatment time-course progression of an existing treatment mathematical model of the targeted biological network. The time-course progression signature can be related to an outcome of the targeted biological network. The new-treatment mathematical model can comprise a new-treatment intervention function modeling each new-treatment intervened-upon node of a set of new-treatment intervened-upon nodes, and a first set of no-treatment node-velocity functions modeling all other nodes of the new-treatment mathematical model.

Each new-treatment intervention function can comprise one more new-treatment intervention constants from a set of new-treatment intervention constants. The existing-treatment mathematical model can comprise an existing-treatment intervention equation modeling each existing-treatment intervened-upon node of a set of existing-treatment intervened-upon nodes, and a no-treatment velocity equation modeling all other nodes of the existing-treatment mathematical model.

Each existing-treatment intervention equation can comprise an existing-treatment intervention constant from a set of existing-treatment intervention constants. The set of new-treatment nodes can be not identical to the set of existing-treatment nodes. Further, the set of new-treatment nodes can comprise at least one node not in the set of existing-treatment nodes. Further, the set of existing-treatment nodes can comprise at least one node not in the set of new-treatment nodes. A drug regimen can be synthesized for each the new treatment intervention constant, and the drug regimens together can be a new drug therapy.

This disclosure teaches new drug therapies designed using any of the afore-mentioned methods. For example, this disclosure teaches a drug therapy having a plurality of drug regimen, each regimen a substance having parameters ascertained using methods described above. Furthermore, the parameters are such that the new drug therapy has an outcome common to an existing drug therapy. Further, the drug regimen each intervene with a node, the nodes together a set of nodes, and such set of nodes are not in common with a second set of nodes intervened upon by the existing drug therapy.

### Drug Development and Design Ecosystem

A new drug development and design application, that can be stored in a memory, can provide properties and characteristics of theoretical therapeutic agent compounds that can be used to identify corresponding real-world potential therapeutic agent compounds. A high-resolution model developed from an identified biochemical or biological network interacting with a known therapeutic agent compound is produced and processed using the drug development and design system. Such method is referred to in this disclosure sometimes as a DASS method. The DASS method can provide a model and information about the effects of the known therapeutic with the identified biochemical or biological network. In one embodiment, the DASS method can also be used in providing models and information about identified biochemical or biological networks without known therapeutics. After the processing using the DASS method, characteristics and properties of theoretical therapeutic agent compounds which affect the identified biochemical or biological network the same way the original therapeutic affects the networks can be identified. Similarly, theoretical therapeutic agents for the particular target cells in the identified biochemical or biological networks without known therapeutics can be identified using the method.

In an embodiment of the DASS method, the system outputs the theoretical properties and characteristics of theoretical therapeutic agent compounds that can be used to interact with the identified biochemical or biological networks. The said properties and characteristics of the theoretical therapeutic agent compounds can be used to compare with real world therapeutic agent compounds and consequently identify potential therapeutic agent compounds, that produces a similar pathway behavior to the biological or biochemical network or at least the target cell, which can be tested on the identified biochemical or biological networks.

In another embodiment, the potential therapeutic agent compounds are simulated against the identified biochemical or biological networks to check if the potential therapeutic agent compounds can be a high-quality drug candidate. A high-quality drug candidate is the identified potential therapeutic agent compound that can produce the same or better effects like the original therapeutic agent compound against a target cell, and causes minimal perturbation on non-target cells, or it produces an overall similar pathway behavior on the identified biochemical or biological network as the original therapeutic does.

In another embodiment, the modelling and simulation of the biochemical or biological networks are performed by the drug development and design system, and all information regarding any modelling performed can be stored in the data storage of the system.

A system to perform the method described herein could comprise a typical networking environment comprising a number of electronic devices and a server connected over a network, including, for example, a system 100. Examples of electronic devices can include, but are not limited to, a computer, a smart phone, and/or a tablet. In one embodiment, electronic device, and server can communicate with each other. Network 107 can be hardwired, wireless, or a combination of both. An example of a LAN is a network within a single building. An example of a WAN is the Internet.

An electronic device can comprise a local memory and a local processor. The local memory can comprise a local application and a local data.

A server can comprise a server memory and a server processor. Server memory can comprise a server application and a server data.

In one embodiment, a drug development and design application can mean local application wherein interface, presentation, logic, and data storage are controlled locally on the electronic device. In such embodiment, memory can mean local memory, processor can mean local processor, and data can mean local data.

In another embodiment, the drug development and design application can mean local application together with server application. One example of such embodiment is where local application is a general-purpose (browser) application. Another example of such embodiment is where local application is a specific-purpose (non-browser) application.

In the first example, a browser accesses the server application via a website. In such embodiment, interfacing with a user would occur using the electronic device, presentation can be performed by local application and server application, while the logic and data can be performed by server. In such example, memory could mean electronic device memory and/or server memory, processor could mean electronic device processor and/or server processor, and data could mean server data.

In the second example, the specific-purpose application accesses server application 103b. In such embodiment, interfacing with the user can occur on electronic device, while presentation, logic, and data storage can be distributed both on electronic device and server. In such example, memory would mean electronic device memory and/or server memory, processor could mean electronic device processor and/or server processor, and data would mean local data and/or server data.

Stored in the memory described herein above are both data and several components that are executable by the processor. In particular, stored in the memory and executable by the processor is the DASS method and potentially other applications. Also stored in the memory can be information such as interaction of known therapeutic agent compounds with target cells, kinetic characterization data of therapeutic agent compounds with non-target cells, and other data. In addition, an operating system can be stored in the memory and executable by the processor.

Although the drug development and design system and other various systems described herein can be embodied in software or code executed by general purpose hardware as discussed above, as an alternative the same can also be embodied in dedicated hardware or a combination of software/general purpose hardware and dedicated hardware. If embodied in dedicated hardware, each can be implemented as a circuit or state machine that employs any one of or a combination of a number of technologies. These technologies can include, but are not limited to, discrete logic circuits having logic gates for implementing various logic functions upon an application of one or more data signals, application specific integrated circuits having appropriate logic gates, or other components, etc. Such technologies are generally well known by those skilled in the art and, consequently, are not described in detail herein.

Also, any logic or application described herein, including the drug development and design system, that comprises software or code can be embodied in any computer-readable storage medium for use by or in connection with an instruction execution system such as, for example, processor in a computer system or other system. In this sense, the logic can comprise, for example, statements including instructions and declarations that can be fetched from the computer-readable storage medium and executed by the instruction execution system.

It should be emphasized that the above-described embodiments of the present disclosure are merely possible examples of implementations set forth for a clear understanding of the principles of the disclosure. Many variations and modifications can be made to the above-described embodiment(s) without departing substantially from the spirit and principles of the disclosure. All such modifications and variations are intended to be included herein within the scope of this disclosure and protected by the following claims.

Various changes in the details of the illustrated operational methods are possible without departing from the scope of the following claims. Some embodiments may combine the activities described herein as being separate steps. Similarly, one or more of the described steps may be omitted, depending upon the specific operational environment the method is being implemented in. It is to be understood that the above description is intended to be illustrative, and not restrictive. For example, the above-described embodiments may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

### Specific Embodiments

The following clauses describe specific embodiments of the disclosure.

Clause 1. A method for developing a new drug therapy using characteristics of an existing drug therapy, the method comprising the following steps: developing a new-treatment mathematical model of a targeted biological network, said new treatment mathematical model capable of producing a new-treatment time-course progression comprising a time-course progression signature found in an existing-treatment time-course progression of an existing treatment mathematical model of said targeted biological network, said time-course progression signature related to an outcome of said targeted biological network; said new-treatment mathematical model comprising a new-treatment intervention function modeling each new-treatment intervened-upon node of a set of new-treatment intervened-upon nodes, and a first set of no-treatment node-velocity functions modeling all other nodes of said new-treatment mathematical model, each said new-treatment intervention function comprising one or new-treatment intervention constants from a set of new-treatment intervention constants; said existing-treatment mathematical model comprising an existing-treatment intervention equation modeling each existing-treatment intervened-upon node of a set of existing-treatment intervened-upon nodes, and a no-treatment velocity equation modeling all other nodes of said existing-treatment mathematical model, each said existing-treatment intervention equation comprising an existing-treatment intervention constant from a set of existing-treatment intervention constants; said set of new-treatment nodes not identical to said set of existing-treatment nodes, further said set of new-treatment nodes comprising at least one node not in said set of existing-treatment nodes, further said set of existing-treatment nodes comprising at least one node not in said set of new-treatment nodes; and synthesizing a drug regimen for each said new treatment intervention constant, said drug regimens together a new drug therapy.

Clause 2. The method of clause 1, wherein said set of new-treatment time-course progressions comprises a new-treatment time-course progression for each chemical of a set of chemicals within said targeted biological network; and said set of existing-treatment time-course progressions comprises an existing-treatment time-course progression for each said chemical of said set of chemicals.

Clause 3. The method of clause 2, wherein said set of new-treatment time-course progressions further comprises a new-treatment time-course progression for each protein of a set of proteins within said targeted biological network; and said set of existing-treatment time-course progressions further comprises an existing-treatment time-course progression for each said protein of said set of proteins.

Clause 4. The method of clause 2, wherein said time-course progression signature comprises a first chemical concentration of a first chemical of said chemicals reaching or passing a threshold.

Clause 5. The method of clause 2, wherein said time-course progression signature comprises a sequence of events.

Clause 6. The method of clause 5, wherein said sequence of events is defined at least in part by a first chemical having a first chemical concentration that meets or passes a first threshold followed by a second chemical having a second chemical concentration that meets or passes a second threshold.

Clause 7. The method of clause 5, wherein the sequence of events is defined at least in part by a first chemical having a first chemical concentration that meets or passes a first threshold followed said first chemical concentration that meets or passes a second threshold.

Clause 8. The method of clause 2, wherein said time-course progression signature comprises a first chemical concentration of a first chemical of said set of chemicals meeting or passing a first threshold while a second chemical concentration of a second chemical of said chemicals is meeting or exceeding a second threshold.

Clause 9. The method of clause 2, wherein said time-course progression signature comprises chemical concentrations of said chemicals of said set of chemicals entering a range such that said chemical concentrations together do not deviate from a set of target concentrations for each of said chemicals by equal to or more than a threshold.

Clause 10. The method of clause 9, wherein a deviation between said chemical concentrations and said set of target concentrations is determined using a root-mean squared calculation.

Clause 11. The method of clause 1, wherein at least one of said new-treatment intervention equations is an inhibition equation and said intervention constant associated with said inhibition equation is an inhibition constant.

Clause 12. The method of clause 1, wherein at least one of said new-treatment intervention equations is an acceleration equation and said intervention constant associated with said acceleration equation is an acceleration constant.

Clause 13. The method of clause 1, wherein at least one of said no-treatment equations is a Michaelis-Menten equation.

Clause 14. The method of clause 1, wherein said set of existing-treatment nodes comprises only one node.

Clause 15. The method of clause 1, wherein there exists no nodes in common with said set of existing treatment nodes and said set of new-treatment nodes.

Clause 16. The method of clause 1, wherein each node in common between said set of existing treatment nodes and said set of new treatment nodes comprise a different existing-treatment intervention constant as compared to a corresponding new-treatment intervention constant.

Clause 17. A method for finding a set of parameters for a new drug therapy such that the new drug therapy produces an outcome similar to an existing drug therapy, comprising the steps: replacing, within a mathematical model any intervention functions related to an existing drug therapy with untreated-node functions related to the mathematical model, if the mathematical model has any such intervention functions; choosing parameter ranges for each of a plurality of parameters; producing a time course progression for a new-treatment mathematical model for each permutation of a plurality of permutations, each time, using said permutation to produce said time course progression; determining for each permutation whether its time-course progression comprises a time-course progression signature present in an existing-treatment time-course progression related to said existing drug therapy, said time-course progression signature related to an outcome of said existing drug therapy; and synthesizing, for at least one permutation comprising said time-course progression signature, substances having kinetic properties substantially matching kinetic parameters of that one permutation, to produce a new drug therapy.

Clause 18. The method of clause 17, wherein said parameter ranges comprise a designation of a set of nodes that can be intervened upon.

Clause 19. The method of clause 17, wherein said parameter ranges comprise a plurality of intervention equations for consideration.

Clause 20. The method of clause 17, wherein parameter ranges comprise a range for one or more intervention constants for one or more intervention equations.

Clause 21. The method of clause 17, wherein parameter ranges comprise intervention constant ranges.

Clause 22. The method of clause 17, wherein parameter ranges comprise spatial considerations.

### Additional Embodiments

Another aspect of the present disclosure provides a computer system comprising: one or more processors; memory; and one or more programs, wherein the one or more programs are stored in the memory and configured to be executed by the one or more processors, the one or more programs including instructions for performing a method for determining an effect of a candidate drug therapy. In some embodiments, the method includes obtaining a reference off-target treatment model for an off-target biological network, where the off-target biological network comprises a first plurality of nodes and a first plurality of biological intermediates, each respective node in the first plurality of nodes representing a respective biological transition between two or more biological intermediates in the first plurality of biological intermediates, the reference off-target treatment model comprises a first subset of intervention nodes and a first subset of control nodes selected from the first plurality of nodes, each respective intervention node in the first subset of intervention nodes comprises (i) a corresponding reference intervention function for the respective biological transition, responsive to a reference drug therapy, at the respective intervention node, and (ii) a corresponding reference intervention constant, in a set of reference intervention constants, for the respective reference intervention function, and each respective control node in the first subset of control nodes comprises a corresponding control function for the respective biological transition, responsive to an absence of the reference drug therapy, at the respective control node. In some embodiments, the method further includes producing, using the reference off-target treatment model, a corresponding reference time-course progression for the off-target biological network comprising at least a first reference signature for a first biological intermediate in the first plurality of biological intermediates. In some embodiments, the method further includes selecting a plurality of candidate drug therapies.

In some embodiments, the method further includes performing, for each respective candidate drug therapy in the plurality of candidate drug therapies, a procedure comprising: determining a respective candidate off-target treatment model for the off-target biological network, where the respective candidate off-target treatment model comprises a second subset of intervention nodes and a second subset of control nodes selected from the first plurality of nodes, each respective intervention node in the second subset of intervention nodes comprises (i) a corresponding candidate intervention function for the respective biological transition, responsive to the respective candidate drug therapy, at the respective intervention node, and (ii) a corresponding candidate intervention constant, in a set of candidate intervention constants, for the respective candidate intervention function, and each respective control node in the second subset of control nodes comprises a corresponding control function for the respective biological transition, responsive to an absence of the respective candidate drug therapy, at the respective control node. In some embodiments, the procedure further includes generating, using the respective candidate off-target treatment model, a corresponding candidate time-course progression for the off-target biological network comprising at least a first candidate signature for the first biological intermediate in the first plurality of biological intermediates.

In some embodiments, the method further includes determining, in the plurality of candidate drug therapies, one or more candidate drug therapies having a corresponding first candidate signature that matches the first reference signature.

Another aspect of the present disclosure provides a computer readable storage medium storing one or more programs, the one or more programs comprising instructions, which when executed by an electronic device with one or more processors and a memory cause the electronic device to perform a method for determining an effect of a candidate drug therapy. In some embodiments, the method includes obtaining a reference off-target treatment model for an off-target biological network, where the off-target biological network comprises a first plurality of nodes and a first plurality of biological intermediates, each respective node in the first plurality of nodes representing a respective biological transition between two or more biological intermediates in the first plurality of biological intermediates, the reference off-target treatment model comprises a first subset of intervention nodes and a first subset of control nodes selected from the first plurality of nodes, each respective intervention node in the first subset of intervention nodes comprises (i) a corresponding reference intervention function for the respective biological transition, responsive to a reference drug therapy, at the respective intervention node, and (ii) a corresponding reference intervention constant, in a set of reference intervention constants, for the respective reference intervention function, and each respective control node in the first subset of control nodes comprises a corresponding control function for the respective biological transition, responsive to an absence of the reference drug therapy, at the respective control node. In some embodiments, the method further includes producing, using the reference off-target treatment model, a corresponding reference time-course progression for the off-target biological network comprising at least a first reference signature for a first biological intermediate in the first plurality of biological intermediates. In some embodiments, the method further includes selecting a plurality of candidate drug therapies.

In some embodiments, the method further includes performing, for each respective candidate drug therapy in the plurality of candidate drug therapies, a procedure comprising: determining a respective candidate off-target treatment model for the off-target biological network, where the respective candidate off-target treatment model comprises a second subset of intervention nodes and a second subset of control nodes selected from the first plurality of nodes, each respective intervention node in the second subset of intervention nodes comprises (i) a corresponding candidate intervention function for the respective biological transition, responsive to the respective candidate drug therapy, at the respective intervention node, and (ii) a corresponding candidate intervention constant, in a set of candidate intervention constants, for the respective candidate intervention function, and each respective control node in the second subset of control nodes comprises a corresponding control function for the respective biological transition, responsive to an absence of the respective candidate drug therapy, at the respective control node. In some embodiments, the procedure further includes generating, using the respective candidate off-target treatment model, a corresponding candidate time-course progression for the off-target biological network comprising at least a first candidate signature for the first biological intermediate in the first plurality of biological intermediates.

In some embodiments, the method further includes determining, in the plurality of candidate drug therapies, one or more candidate drug therapies having a corresponding first candidate signature that matches the first reference signature.

Another aspect of the present disclosure provides a computer system comprising: one or more processors; memory; and one or more programs, wherein the one or more programs are stored in the memory and configured to be executed by the one or more processors, the one or more programs including instructions for performing any of the methods and/or embodiments disclosed herein.

Another aspect of the present disclosure provides a computer readable storage medium storing one or more programs, the one or more programs comprising instructions, which when executed by an electronic device with one or more processors and a memory cause the electronic device to perform any of the methods and/or embodiments disclosed herein.

### Conclusion

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that comprises a computer program mechanism embedded in a non-transitory computer readable storage medium. For instance, the computer program product could contain the program modules shown in any combination of FIGS. 21A-C or 22A-E. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed is:

1. A method for determining an effect of a candidate drug therapy, the method comprising,
at a computer system comprising one or more processors and a memory:
(A) obtaining a reference off-target treatment model for an off-target biological network, wherein:
the off-target biological network comprises a first plurality of nodes and a first plurality of biological intermediates, each respective node in the first plurality of nodes representing a respective biological transition between two or more biological intermediates in the first plurality of biological intermediates,
the reference off-target treatment model comprises a first subset of intervention nodes and a first subset of control nodes selected from the first plurality of nodes,
each respective intervention node in the first subset of intervention nodes comprises (i) a corresponding reference intervention function for the respective biological transition, responsive to a reference drug therapy, at the respective intervention node, and (ii) a corresponding reference intervention constant, in a set of reference intervention constants, for the respective reference intervention function, and
each respective control node in the first subset of control nodes comprises a corresponding control function for the respective biological transition, responsive to an absence of the reference drug therapy, at the respective control node;
(B) producing, using the reference off-target treatment model, a corresponding reference time-course progression for the off-target biological network comprising at least a first reference signature for a first biological intermediate in the first plurality of biological intermediates;
(C) selecting a plurality of candidate drug therapies;
(D) performing, for each respective candidate drug therapy in the plurality of candidate drug therapies, a procedure comprising:
determining a respective candidate off-target treatment model for the off-target biological network, wherein:
the respective candidate off-target treatment model comprises a second subset of intervention nodes and a second subset of control nodes selected from the first plurality of nodes,
each respective intervention node in the second subset of intervention nodes comprises (i) a corresponding candidate intervention function for the respective biological transition, responsive to the respective candidate drug therapy, at the respective intervention node, and (ii) a corresponding candidate intervention constant, in a set of candidate intervention constants, for the respective candidate intervention function, and
each respective control node in the second subset of control nodes comprises a corresponding control function for the respective biological transition, responsive to an absence of the respective candidate drug therapy, at the respective control node, and
generating, using the respective candidate off-target treatment model, a corresponding candidate time-course progression for the off-target biological network comprising at least a first candidate signature for the first biological intermediate in the first plurality of biological intermediates; and
(E) determining, in the plurality of candidate drug therapies, one or more candidate drug therapies having a corresponding first candidate signature that matches the first reference signature.

2. The method of claim 1, wherein the first plurality of biological intermediates comprises at least 3 biological intermediates.

3. The method of claim 1 or 2, wherein each respective biological intermediate in the first plurality of biological intermediates is a polypeptide or a nucleic acid.

4. The method of any one of claims 1-3, wherein the first plurality of nodes comprises at least 5 nodes.

5. The method of any one of claims 1-4, wherein, for a respective node in the first plurality of nodes, the respective biological transition is a chemical interconversion or a regulatory function.

6. The method of any one of claims 1-5, wherein the off-target biological network is selected from the group consisting of: an organism type, a disease condition, a cell type, and a biological pathway.

7. The method of any one of claims 1-6, wherein the reference drug therapy is selected as a treatment for a targeted biological network.

8. The method of claim 7, wherein the targeted biological network is for a first organism, and the off-target biological network is for a second organism.

9. The method of claim 8, wherein the first organism is a pathogen, and the second organism is a host organism.

10. The method of claim 9, wherein the pathogen is E. coli.

11. The method of claim 9 or 10, wherein the host organism is a human.

12. The method of claim 7, wherein the targeted biological network is a disease cell and the off-target biological network is a healthy cell.

13. The method of claim 7, wherein the targeted biological network is a cancer cell and the off-target biological network is a normal cell.

14. The method of claim 7, wherein the targeted biological network is a first cell type and the off-target biological network is a second cell type.

15. The method of claim 7, wherein the targeted biological network is a first biological pathway and the off-target biological network is a second biological pathway, other than the first biological pathway.

16. The method of any one of claims 1-15, further comprising repeating the obtaining A), producing B), selecting C), and performing D) for each off-target biological network in a plurality of off-target biological networks.

17. The method of claim 16, wherein each respective off-target biological network in the plurality of off-target biological networks has a different biological condition selected from the group consisting of: organism type, disease condition, cell type, and biological pathway.

18. The method of any one of claims 1-17, wherein, for a respective intervention node in the first subset of intervention nodes, the corresponding reference intervention function is an inhibition function.

19. The method of any one of claims 1-18, wherein, for a respective intervention node in the first subset of intervention nodes, the corresponding reference intervention constant is an inhibition constant.

20. The method of any one of claims 1-19, wherein, for a respective intervention node in the first subset of intervention nodes, the corresponding reference intervention function is an acceleration function.

21. The method of any one of claims 1-20, wherein, for a respective intervention node in the first subset of intervention nodes, the corresponding reference intervention constant is an acceleration constant.

22. The method of any one of claims 1-21, wherein, for each respective intervention node in the first subset of intervention nodes, the corresponding reference intervention function is a Michaelis-Menten equation.

23. The method of any one of claims 1-22, wherein the first subset of intervention nodes comprises at least 1 intervention node.

24. The method of any one of claims 1-22, wherein the first subset of intervention nodes consists of only one node.

25. The method of any one of claims 1-24, wherein, for each respective control node in the first subset of control nodes, the corresponding control function is a Michaelis-Menten equation.

26. The method of any one of claims 1-25, wherein the first reference signature for the first biological intermediate in the first plurality of biological intermediates comprises, for each timepoint in a plurality of timepoints, a corresponding concentration of the first biological intermediate.

27. The method of any one of claims 1-26, wherein, for each respective candidate drug therapy in the plurality of candidate drug therapies, the second subset of intervention nodes comprises at least one node that is not contained in the first subset of intervention nodes.

28. The method of any one of claims 1-27, wherein, for each respective candidate drug therapy in the plurality of candidate drug therapies, the first subset of intervention nodes comprises at least one node that is not contained in the second subset of intervention nodes.

29. The method of any one of claims 1-28, wherein the first candidate signature for the first biological intermediate in the first plurality of biological intermediates comprises, for each timepoint in a plurality of timepoints, a corresponding concentration of the first biological intermediate.

30. The method of any one of claims 1-29, further comprising (F) synthesizing, for each respective candidate drug therapy having a corresponding first candidate signature that matches the first reference signature, the respective candidate drug therapy.

31. The method of claim 30, further comprising, after the synthesizing F) using the one or more candidate drug therapies as treatment for a target biological network.

32. The method of any one of claims 1-31, wherein the reference drug therapy is an existing drug therapy for a targeted biological network, further comprising determining a candidate drug therapy by a process comprising:
(A) obtaining an existing-treatment mathematical model for the targeted biological network comprising a plurality of nodes, wherein:
the existing-treatment mathematical model comprises:
a first subset of intervened-upon nodes in the plurality of nodes, each respective intervened-upon node in the first subset of intervened-upon nodes comprising (i) a corresponding existing-treatment intervention function that represents one or more chemical interconversions, responsive to the existing drug therapy, at the respective intervened-upon node and (ii) a corresponding existing-treatment intervention constant, from a set of existing-treatment intervention constants, for the respective existing-treatment intervention function, and
a second subset of no-treatment nodes in the plurality of nodes, each respective no-treatment node in the second subset of no-treatment nodes comprising a corresponding no-treatment function that represents one or more chemical interconversions, responsive to an absence of the existing drug therapy, at the respective no-treatment node, and
the existing-treatment mathematical model produces a corresponding existing-treatment time-course progression for the targeted biological network that comprises at least a first signature that is related to an existing-treatment outcome of the targeted biological network;
(B) developing, for each respective permutation in a plurality of permutations, a respective new-treatment mathematical model of the targeted biological network, wherein:
the respective new treatment mathematical model comprises:
a third subset of intervened-upon nodes in the plurality of nodes, each respective intervened-upon node in the third subset of intervened-upon nodes comprising (i) a corresponding new-treatment intervention function that represents one or more chemical interconversions at the respective intervened-upon node and (ii) a corresponding new-treatment intervention constant, from a corresponding set of new-treatment intervention constants, for the respective new-treatment intervention function, and
a fourth subset of no-treatment nodes in the plurality of nodes, each respective no-treatment node in the fourth subset of no-treatment nodes comprising a corresponding no-treatment function that represents one or more chemical interconversions at the respective no-treatment node,
the respective new-treatment mathematical model produces a corresponding new-treatment time-course progression for the targeted biological network comprising at least a corresponding second signature related to a respective new-treatment outcome of the targeted biological network,
the first subset of intervened-upon nodes comprises at least one node not in the third subset of intervened-upon nodes, and
the third subset of intervened-upon nodes comprises at least one node not in the first subset of intervened-upon nodes; and
(C) synthesizing, for each respective permutation in the plurality of permutations having a corresponding second signature that matches the first signature for the existing-treatment time-course progression, a corresponding drug regimen for each respective new treatment intervention constant in the corresponding set of new-treatment intervention constants,
thereby obtaining a set of drug regimens together forming a new drug therapy for the respective permutation, wherein at least one drug regimen in the set of drug regimens is not included in the existing drug therapy.

33. A computer system comprising:
one or more processors;
memory; and
one or more programs, wherein the one or more programs are stored in the memory and configured to be executed by the one or more processors, the one or more programs including instructions for performing a method comprising
(A) obtaining a reference off-target treatment model for an off-target biological network, wherein:
the off-target biological network comprises a first plurality of nodes and a first plurality of biological intermediates, each respective node in the first plurality of nodes representing a respective biological transition between two or more biological intermediates in the first plurality of biological intermediates,
the reference off-target treatment model comprises a first subset of intervention nodes and a first subset of control nodes selected from the first plurality of nodes,
each respective intervention node in the first subset of intervention nodes comprises (i) a corresponding reference intervention function for the respective biological transition, responsive to a reference drug therapy, at the respective intervention node, and (ii) a corresponding reference intervention constant, in a set of reference intervention constants, for the respective reference intervention function, and
each respective control node in the first subset of control nodes comprises a corresponding control function for the respective biological transition, responsive to an absence of the reference drug therapy, at the respective control node;
(B) producing, using the reference off-target treatment model, a corresponding reference time-course progression for the off-target biological network comprising at least a first reference signature for a first biological intermediate in the first plurality of biological intermediates;
(C) selecting a plurality of candidate drug therapies;
(D) performing, for each respective candidate drug therapy in the plurality of candidate drug therapies, a procedure comprising:
determining a respective candidate off-target treatment model for the off-target biological network, wherein:
the respective candidate off-target treatment model comprises a second subset of intervention nodes and a second subset of control nodes selected from the first plurality of nodes,
each respective intervention node in the second subset of intervention nodes comprises (i) a corresponding candidate intervention function for the respective biological transition, responsive to the respective candidate drug therapy, at the respective intervention node, and (ii) a corresponding candidate intervention constant, in a set of candidate intervention constants, for the respective candidate intervention function, and
each respective control node in the second subset of control nodes comprises a corresponding control function for the respective biological transition, responsive to an absence of the respective candidate drug therapy, at the respective control node, and
generating, using the respective candidate off-target treatment model, a corresponding candidate time-course progression for the off-target biological network comprising at least a first candidate signature for the first biological intermediate in the first plurality of biological intermediates; and
(E) determining, in the plurality of candidate drug therapies, one or more candidate drug therapies having a corresponding first candidate signature that matches the first reference signature.

34. A non-transitory computer readable storage medium storing one or more programs, the one or more programs comprising instructions, which when executed by an electronic device with one or more processors and a memory cause the electronic device to perform a method comprising:
(A) obtaining a reference off-target treatment model for an off-target biological network, wherein:
the off-target biological network comprises a first plurality of nodes and a first plurality of biological intermediates, each respective node in the first plurality of nodes representing a respective biological transition between two or more biological intermediates in the first plurality of biological intermediates,
the reference off-target treatment model comprises a first subset of intervention nodes and a first subset of control nodes selected from the first plurality of nodes,
each respective intervention node in the first subset of intervention nodes comprises (i) a corresponding reference intervention function for the respective biological transition, responsive to a reference drug therapy, at the respective intervention node, and (ii) a corresponding reference intervention constant, in a set of reference intervention constants, for the respective reference intervention function, and
each respective control node in the first subset of control nodes comprises a corresponding control function for the respective biological transition, responsive to an absence of the reference drug therapy, at the respective control node;
(B) producing, using the reference off-target treatment model, a corresponding reference time-course progression for the off-target biological network comprising at least a first reference signature for a first biological intermediate in the first plurality of biological intermediates;
(C) selecting a plurality of candidate drug therapies;
(D) performing, for each respective candidate drug therapy in the plurality of candidate drug therapies, a procedure comprising:
determining a respective candidate off-target treatment model for the off-target biological network, wherein:
the respective candidate off-target treatment model comprises a second subset of intervention nodes and a second subset of control nodes selected from the first plurality of nodes,
each respective intervention node in the second subset of intervention nodes comprises (i) a corresponding candidate intervention function for the respective biological transition, responsive to the respective candidate drug therapy, at the respective intervention node, and (ii) a corresponding candidate intervention constant, in a set of candidate intervention constants, for the respective candidate intervention function, and
each respective control node in the second subset of control nodes comprises a corresponding control function for the respective biological transition, responsive to an absence of the respective candidate drug therapy, at the respective control node, and
generating, using the respective candidate off-target treatment model, a corresponding candidate time-course progression for the off-target biological network comprising at least a first candidate signature for the first biological intermediate in the first plurality of biological intermediates; and
(E) determining, in the plurality of candidate drug therapies, one or more candidate drug therapies having a corresponding first candidate signature that matches the first reference signature.
